(19) United States
(12) Patent Application Publication
(10) Pub. No.: US 2012/0046331 A1
(43) Pub. Date: Feb. 23, 2012

(54) Antifungal Agents and Uses Thereof

(76) Inventor: Jeffrey M. Besterman, Baie d'Urfe (CA)

(21) Appl. No.: 13/213,372

(22) Filed: Aug. 19, 2011

Related U.S. Application Data
(60) Provisional application No. 61/375,435, filed on Aug. 20, 2010.

(51) Int. Cl. A61K 31/185; A61K 31/351; A61P 31/10; C07D 209/08; C07D 309/12; A61K 31/404; C07C 259/10

(52) U.S. Cl. 514/415; 514/575; 514/459; 562/622; 548/469; 549/419

(57) ABSTRACT

This invention relates to new antifungal agents, compositions thereof, and methods for inhibiting the growth of fungi involved in infection and disease of keratinized tissue, such as onychomycosis. The invention also relates to new antifungal agents, compositions thereof, and methods for treating and/or preventing fungal infection and/or disease of keratinized tissue, such as onychomycosis. The invention further relates to a kit comprising said antifungal agent and use of said kit in treatment of fungal infection and/or disease of keratinized tissue, such as onychomycosis.

### BACKGROUND OF THE INVENTION

1. Field of the Invention

This invention relates to new antifungal agents, compositions thereof, and methods for inhibiting the growth of fungi involved in infection and disease of keratinized tissue, such as onychomycosis. The invention also relates to new antifungal agents, compositions thereof, and methods for treating and/or preventing fungal infection and/or disease of keratinized tissue, such as onychomycosis. The invention further relates to a kit comprising said antifungal agent and use of said kit in treatment of fungal infection and/or disease of keratinized tissue, such as onychomycosis.

2. Description of Related Art

Dermatophytoses are fungal infections of keratinized tissue (such as skin, hair, nails, and claws). Such fungal infections can be caused by different fungal species, such as Epidermophyton, Microsporum and Trichophyton spp., collectively known as dermatophytes. These pathogenic fungi are found throughout the world and can infect and cause disease in humans and other animals, such as domesticated animals. Some dennatophyte species, such as M. gypseum and T. terrestre, inhabit the soil (geophilic), and cause disease after exposure to infected soil. Other species, such as M. audouinii and T. rubrum, are host-adapted to humans (anthropophilic), and infect other animals rarely. The most important animal pathogens worldwide are M. canis, M. gypseum, T. mentagrophytes, T. equinum, T. verrucosum, and M. nanum. These species can be spread to people, especially M. canis infections of domestic cats and T. verrucosum of cattle. Treatment methods vary but involve either topical or oral administration of antifungal drugs.

Onychomycosis, one of the most common nail disorders, is caused by fungal infection of the nail plate, nail bed, or both. About 60 to 80% of cases are caused by dermatophytes (Trichophyton rubrum, T. mentagrophytes and Epidermophyton floccosum are the most common etiologic agents worldwide); dermatophyte infection of the nails is called tinea unguium. Many of the remaining cases are caused by nondermatophyte molds (eg, Aspergillus, Scopulariopsis, and Fusarium). Baudraz-Rosselet et al. (2010, Dermatology 220(2):164-168) for example report that Fusarium sp., Acremonium sp. and Aspergillus sp. were found as a sole infectious agent in certain cases. Immunocompromised patients and those with chronic mucocutaneous candidiasis may have candidal onychomycosis (which is more common among the fingers). The leading non-dermatophyte mould cause of onychomycosis is Scopulariopsis brevicaulis. The leading yeast cause of onychomycosis is Candida albicans.

The use of, and need for, antifungal agents is widespread and ranges from the treatment of mycotic infections in animals; to disinfectant formulations; to pharmaceuticals for human use. A major problem with current antifungal formulations is their toxicity to the infected host. This is particularly important in cases where many fungal infestations are opportunistic infections secondary to debilitating diseases, such as AIDS or from cancer chemotherapy or organ transplants. Correspondingly, at least for antifungal agents that are to be administered to humans and other animals, the therapeutic index is preferably such that toxicity is selective to the targeted fungus without being toxic to the host.

Drawbacks to current antifungal agents, such as the azoles, include development of resistance, possible drug-drug interactions and possible toxic liver effects.

It would be highly desirable to be provided with new antifungal agents, compositions thereof, and methods for inhibiting the growth of fungi involved in infection and/or disease of keratinized tissue, such as onychomycosis. It would also be highly desirable to be provided with new antifungal agents, compositions thereof, and methods for treating and/or preventing fungal infection and/or disease of keratinized tissue, such as onychomycosis. It would also be highly desirable to provide such new antifungal agents, compositions thereof, and methods which are selectively toxic to the pathological fungus without being toxic to the host.

### SUMMARY OF THE INVENTION

It has been surprisingly found that certain compounds have antifungal activity and show synergistic activity with other antifungal agents against fungal species involved in infection and/or disease of keratinized tissue, such as onychomycosis. In certain embodiments of aspects of the present invention the compounds are inhibitors of histone deacetylase.

In a first aspect, the present invention provides a method for inhibiting the growth of a fungus or fungal unit thereof involved in infection and/or disease of keratinized tissue, such as onychomycosis, comprising contacting the fungus or fungal unit thereof with a growth inhibiting effective amount of a compound according to the present invention.

In a second aspect, the present invention provides a method for inhibiting the growth of a fungus or fungal unit thereof involved in infection and/or disease of keratinized tissue, such as onychomycosis, comprising inhibiting the activity of a histone deacetylase in the fungus or fungal unit thereof.

In a third aspect, the present invention provides a method for treating and/or preventing a fungal infection and/or disease of keratinized tissue, such as onychomycosis, in a subject comprising administering to the subject in need thereof a treatment or preventative effective amount of a compound according to the present invention.

In a fourth aspect, the present invention provides a method for treating and/or preventing a fungal infection and/or disease of keratinized tissue, such as onychomycosis, in a subject, comprising inhibiting the activity of a histone deacetylase in the fungus or a fungal unit thereof.

In a fifth aspect, the present invention provides a method for sensitizing a fungus or fungal unit thereof involved in infection and/or disease of keratinized tissue, such as onychomycosis, to an antifungal compound, comprising contacting the fungus or fungal unit thereof with a sensitizing effective amount of a compound according to the present invention.

In a sixth aspect, the present invention provides a method for sensitizing a fungus or fungal unit thereof involved in infection and/or disease of keratinized tissue, such as onychomycosis, to an antifungal compound, comprising inhibiting the activity of a histone deacetylase in the fungus or fungal unit thereof.

In a seventh aspect, the present invention provides a method for enhancing the activity of an antifungal agent against a fungus or fungal unit thereof involved in infection and/or disease of keratinized tissue, such as onychomycosis, comprising contacting the fungus or fungal unit thereof with the antifungal agent in combination with an activity enhancing effective amount of a compound according to the present invention.

In an eighth aspect, the present invention provides a method for enhancing the activity of an antifungal agent against a fungus or fungal unit thereof involved in infection and/or disease of keratinized tissue, such as onychomycosis, comprising contacting the fungus or fungal unit thereof with the antifungal agent in combination with inhibiting the activity of a histone deacetylase in the fungus or fungal unit thereof.

In a ninth aspect, the present invention provides a kit, comprising a compound according to the present invention, and optionally instructions for using the kit in a method according to the present invention.

In a tenth aspect, the present invention provides a kit, comprising inhibitor of fungal histone deacetylase, and optionally instructions for using the kit in a method according to the present invention.

It will be understood by those of skill in the art that in the methods and uses of the present invention, compositions comprising a compound according to the present invention, or an inhibitor of fungal histone deacetylase, and a pharmaceutically acceptable carrier, excipient or diluent, may be used in place of the compound or inhibitor itself, respectively.

Because the invention provides compounds, for example compounds of Formula (I), Formula (Ia) and Formula (II), and prodrugs of Formula (Ib) and Formula (IIa), which are useful in aspects of the present invention, such compounds, therefore, are useful research tools for the in vitro and/or in vivo study of fungi involved in infection and/or disease of keratinized tissue, such as onychomycosis.

The foregoing merely summarizes certain aspects of the invention and is not intended to be limiting in nature. These aspects and other aspects and embodiments are described more fully below. The patent and scientific literature referred to herein establishes knowledge that is available to those with skill in the art. The issued patents, applications, and references that are cited herein are hereby incorporated by reference to the same extent as if each was specifically and individually indicated to be incorporated by reference. In the case of inconsistencies, the present disclosure will prevail.

### DETAILED DESCRIPTION OF THE INVENTION

It has been surprisingly found that certain compounds have antifungal activity and show synergistic activity with other antifungal agents against fungal species involved in infection and/or disease of keratinized tissue, such as onychomycosis. In certain embodiments of the present invention the compounds are inhibitors of histone deacetylase.

In a first aspect of the present invention, the present invention provides a method for inhibiting the growth of a fungus or fungal unit thereof involved in infection and/or disease of keratinized tissue, such as onychomycosis, comprising contacting the fungus or fungal unit thereof with a growth inhibiting effective amount of a compound according to the present invention.

In certain embodiments of the first aspect, the method is an in vitro method for inhibiting the growth of the fungus or fungal unit thereof. In certain embodiments, the method is an in vivo method for inhibiting the growth of the fungus or fungal unit thereof. If in vivo, the method comprises administering to a subject having growth of the fungus or fungal unit thereof thereon and/or therein, a growth inhibiting effective amount of a compound according to the present invention. In certain embodiments of the first aspect, the growth inhibiting effect of the compound is more active against the fungus or fungal unit thereof than against a human or other animal cell. In certain embodiments, the growth inhibiting effect of the compound is specific for the fungus or fungal unit thereof.

In certain embodiments of the first aspect, the compounds according to the present invention may also be combined with another antifungal agent to form an antifungal mixture or a synergistic mixture thereof. Accordingly, in certain embodiments of the first aspect, the method further comprises contacting the fungus or fungal unit thereof with another antifungal agent, or, if in vivo, administering to the subject another antifungal agent. In certain embodiments of the first aspect, the compound according to the present invention and the other antifungal agent are in respective proportions to provide a synergistic effect to inhibit the growth of the fungus or fungal unit thereof when compared to either the compound according to the present invention alone or the other antifungal agent alone. As will be understood by those skilled in the art, the synergistic effect may be obtained within various proportions of the compound according to the present invention and the other antifungal agent, depending for example on the kind of fungus or fungal unit thereof towards which effect is measured. When used in combination with another antifungal agent(s), a compound according to the present invention can be formulated together with the other antifungal agent(s) co-administered with the other antifungal agents(s), or applied sequentially the other antifungal agent(s).

In accordance with certain embodiments of the first aspect, the present invention provides compounds and synergistic combinations of said compounds and another antifungal agent, which inhibit the growth of a fungus or fungal unit thereof involved in infection and/or disease of keratinized tissue, such as onychomycosis. In accordance with certain embodiments of the first aspect, the invention also provides for the use of a compound according to the present invention and a synergistic combination of said compound and another antifungal agent, for inhibiting the growth of a fungus or fungal unit thereof involved in infection and/or disease of keratinized tissue, such as onychomycosis. Also in accordance with certain embodiments of the first aspect, the invention provides for the use of a compound according to the present invention or a synergistic combination of said compound and another antifungal agent, in the manufacture of a medicament to inhibit the growth of a fungus or fungal unit thereof involved in infection and/or disease of keratinized tissue, such as onychomycosis.

In a second aspect, the present invention provides a method for inhibiting the growth of a fungus or fungal unit thereof involved in infection and/or disease of keratinized tissue, such as onychomycosis, comprising inhibiting the activity of a histone deacetylase in the fungus or fungal unit thereof.

In certain embodiments of the second aspect, the method is an in vitro method for inhibiting the growth of the fungus or fungal unit thereof. If in vitro, inhibiting the activity of a histone deacetylase in the fungus or fungal unit thereof comprises contacting the fungus or fungal unit thereof with a growth inhibiting effective amount of an inhibitor of the activity of a histone deacetylase in the fungus or fungal unit thereof. In certain embodiments, the method is an in vivo method for inhibiting the growth of the fungus or fungal unit thereof. If in vivo, inhibiting the activity of a histone deacetylase in the fungus or fungal unit thereof comprises administering to a subject having growth of the fungus or fungal unit thereof thereon and/or therein, a growth inhibiting effective amount of an inhibitor of the activity of a histone deacetylase in the fungus or fungal unit thereof.

In certain embodiments of the second aspect, the inhibitor of the activity of a histone deacetylase in the fungus or fungal unit thereof may also be combined with another antifungal agent to form an antifungal mixture or a synergistic mixture thereof. Accordingly, in certain embodiments of the second aspect, the method further comprises contacting the fungus or fungal unit thereof with another antifungal agent, or, if in vivo, administering to the subject another antifungal agent. In certain embodiments of the second aspect, the inhibitor of the activity of a histone deacetylase in the fungus or fungal unit thereof and the other antifungal agent are in respective proportions to provide a synergistic effect to inhibit the growth of the fungus or fungal unit thereof when compared to either the inhibitor of the activity of a histone deacetylase in the fungus or fungal unit thereof alone or the other antifungal agent alone. As will be understood by those skilled in the art, the synergistic effect may be obtained within various proportions of the inhibitor of the activity of a histone deacetylase in the fungus or fungal unit thereof and the other antifungal agent, depending for example on the kind of fungus or fungal unit thereof towards which effect is measured. When used in combination with another antifungal agent(s), the inhibitor of the activity of a histone deacetylase in the fungus or fungal unit thereof can be formulated together with the other antifungal agent(s), co-administered with the other antifungal agent(s), or applied sequentially with the other antifungal agent(s).

In accordance with certain embodiments of the second aspect, the present invention provides inhibitors of the activity of a histone deacetylase in a fungus or fungal unit thereof and synergistic combinations of said inhibitors and another antifungal agent, which inhibit the growth of a fungus or fungal unit thereof involved in infection and/or disease of keratinized tissue, such as onychomycosis. In accordance with certain embodiments of the second aspect, the invention also provides for the use of an inhibitor of the activity of a histone deacetylase in a fungus or fungal unit thereof and a synergistic combination of said inhibitor and another antifungal agent, for inhibiting the growth of a fungus or fungal unit thereof involved in infection and/or disease of keratinized tissue, such as onychomycosis. Also in accordance with certain embodiments of the second aspect, the invention provides for the use of an inhibitor of the activity of a histone deacetylase in a fungus or fungal unit thereof or a synergistic combination of said inhibitor and another antifungal agent, in the manufacture of a medicament to inhibit the growth of a fungus or fungal unit thereof involved in infection and/or disease of keratinized tissue, such as onychomycosis.

In a third aspect, the present invention provides a method for treating and/or preventing a fungal infection and/or disease of keratinized tissue, such as onychomycosis, in a subject comprising administering to the subject in need thereof a treatment or preventative effective amount of a compound according to the present invention.

In certain embodiments of the third aspect, the compounds according to the present invention may also be combined with another antifungal agent to form an antifungal mixture or a synergistic mixture thereof. Accordingly, in certain embodiments of the third aspect, the method further comprises administering to the subject another antifungal agent. In certain embodiments of the third aspect, the compound according to the present invention and the other antifungal agent are in respective proportions to provide a synergistic effect to treat and/or prevent a fungal infection and/or disease of keratinized tissue, such as onychomycosis, when compared to either the compound according to the present invention alone or the other antifungal agent alone. As will be understood by those skilled in the art, the synergistic effect may be obtained within various proportions of the compound according to the present invention and the other antifungal agent, depending for example on the kind of fungus or fungal unit thereof towards which effect is measured. When used in combination with another antifungal agent(s), the compounds according to the present invention can be formulated together with the other antifungal agent(s), co-administered with other antifungal agent(s), or applied sequentially with the other antifungal agent(s).

In accordance with certain embodiments of the third aspect, the present invention provides compounds and synergistic combinations of said compounds and another antifungal agent, for treating and/or preventing a fungal infection and/or disease of keratinized tissue, such as onychomycosis, in a subject. In accordance with certain embodiments of the third aspect, the invention also provides for the use of a compound according to the present invention and a synergistic combination of said compound and another antifungal agent, for treating and/or preventing a fungal infection and/or disease of keratinized tissue, such as onychomycosis, in a subject. Also in accordance with certain embodiments of the third aspect, the invention provides for the use of a compound according to the present invention or a synergistic combination of said compound and another antifungal agent, in the manufacture of a medicament to treat and/or prevent a fungal infection and/or disease of keratinized tissue, such as onychomycosis, in a subject.

In certain embodiments of the third aspect, the method is a method for treating a fungal infection and/or disease of keratinized tissue, such as onychomycosis, in a subject.

In certain embodiments of the third aspect, the method is a method for preventing a fungal infection and/or disease of keratinized tissue, such as onychomycosis, in a subject.

In certain embodiments of the third aspect, the antifungal effect of the compound is greater than its effect against a human or other animal cell. In certain embodiments, the activity of the compound is specific for the fungus or fungal unit thereof.

In a fourth aspect, the present invention provides a method for treating and/or preventing a fungal infection and/or disease of keratinized tissue, such as onychomycosis, in a subject, comprising inhibiting the activity of a histone deacetylase in the fungus or a fungal unit thereof.

In certain embodiments of the fourth aspect, inhibiting the activity of a histone deacetylase in the fungus or fungal unit thereof comprises administering to a subject having growth of the fungus or fungal unit thereof thereon and/or therein, a treatment and/or preventative effective amount of an inhibitor of the activity of a histone deacetylase in the fungus or fungal unit thereof.

In certain embodiments of the fourth aspect, the inhibitor of the activity of a histone deacetylase in the fungus or fungal unit thereof may also be combined with another antifungal agent to form an antifungal mixture or a synergistic mixture thereof. Accordingly, in certain embodiments of the fourth aspect, the method further comprises administering to the subject another antifungal agent. In certain embodiments of the fourth aspect, the inhibitor of the activity of a histone deacetylase in the fungus or fungal unit thereof and the other antifungal agent are in respective proportions to provide a synergistic effect to treat and/or prevent a fungal infection and/or disease of keratinized tissue, such as onychomycosis when compared to either the inhibitor of the activity of a histone deacetylase in the fungus or fungal unit thereof alone or the other antifungal agent alone. As will be understood by those skilled in the art, the synergistic effect may be obtained within various proportions of the inhibitor of the activity of a histone deacetylase in the fungus or fungal unit thereof and the other antifungal agent, depending for example on the kind of fungus or fungal unit thereof towards which effect is measured. When used in combination with another antifungal agent(s), the inhibitor of the activity of a histone deacetylase in the fungus or fungal unit thereof can be formulated together with the other antifungal agent(s), co-administered with the other antifungal agent(s), or applied sequentially with the other antifungal agent(s).

In accordance with certain embodiments of the fourth aspect, the present invention provides inhibitors of the activity of a histone deacetylase in a fungus or fungal unit thereof and synergistic combinations of said inhibitors and another antifungal agent, for treating and/or preventing a fungal infection and/or disease of keratinized tissue, such as onychomycosis, in a subject. In accordance with certain embodiments of the fourth aspect, the invention also provides for the use of an inhibitor of the activity of a histone deacetylase in a fungus or fungal unit thereof and a synergistic combination of said inhibitor and another antifungal agent, for treating and/or preventing a fungal infection and/or disease of keratinized tissue, such as onychomycosis, in a subject. Also in accordance with certain embodiments of the fourth aspect, the invention provides for the use of an inhibitor of the activity of a histone deacetylase in a fungus or fungal unit thereof or a synergistic combination of said inhibitor and another antifungal agent, in the manufacture of a medicament to treat and/or prevent a fungal infection and/or disease of keratinized tissue, such as onychomycosis, in a subject.

In a fifth aspect, the present invention provides a method for sensitizing a fungus or fungal unit thereof involved in infection and/or disease of keratinized tissue, such as onychomycosis, to an antifungal agent, comprising contacting the fungus or fungal unit thereof with a sensitizing effective amount of a compound according to the present invention.

In certain embodiments of the fifth aspect, the method is an in vitro method for sensitizing a fungus or fungal unit thereof involved in infection and/or disease of keratinized tissue, such as onychomycosis, to an antifungal compound. In certain embodiments, the method is an in vivo method for sensitizing a fungus or fungal unit thereof involved in infection and/or disease of keratinized tissue, such as onychomycosis, to an antifungal compound. If in vivo, the method comprises administering to a subject having growth of the fungus or fungal unit thereof thereon and/or therein, a sensitizing effective amount of a compound according to the present invention.

In certain embodiments of the fifth aspect, the compounds according to the present invention may be combined with the antifungal agent to form an antifungal mixture or a synergistic mixture thereof. Accordingly, in certain embodiments of the fifth aspect, the method further comprises contacting the fungus or fungal unit thereof with the antifungal agent, or, if in vivo, administering to the subject the antifungal agent. In certain embodiments of the fifth aspect, the compound according to the present invention and the antifungal agent are in respective proportions to provide a synergistic antifungal effect against the fungus or fungal unit thereof when compared to either the compound according to the present invention alone or the antifungal agent alone. As will be understood by those skilled in the art, the synergistic effect may be obtained within various proportions of the compound according to the present invention and the antifungal agent, depending for example on the kind of fungus or fungal unit thereof towards which effect is measured. When used in combination with an antifungal agent(s), the compounds according to the present invention can be formulated together with the antifungal agent(s), co-administered with the other antifungal agent(s), or applied sequentially with the antifungal agent(s).

In certain embodiments of the fifth aspect, the sensitizing effect of the compound on the fungus or fungal unit thereof is greater than its effect on a human or other animal cell. In certain embodiments, the sensitizing effect of the compound is specific for the fungus or fungal unit thereof.

In accordance with certain embodiments of the fifth aspect, the present invention provides compounds and synergistic combinations of said compounds and an antifungal agent, for sensitizing a fungus or fungal unit thereof involved in infection and/or disease of keratinized tissue, such as onychomycosis, to the antifungal agent. In accordance with certain embodiments of the fifth aspect, the invention also provides for the use of a compound according to the present invention and a synergistic combination of said compound and an antifungal agent, for sensitizing a fungus or fungal unit thereof involved in infection and/or disease of keratinized tissue, such as onychomycosis, to the antifungal agent. Also in accordance with certain embodiments of the fifth aspect, the invention provides for the use of a compound according to the present invention or a synergistic combination of said compound and an antifungal agent, in the manufacture of a medicament to sensitize a fungus or fungal unit thereof involved in infection and/or disease of keratinized tissue, such as onychomycosis, to the antifungal agent.

In a sixth aspect, the present invention provides a method for sensitizing a fungus or fungal unit thereof involved in infection and/or disease of keratinized tissue, such as onychomycosis, to an antifungal agent, comprising inhibiting the activity of a histone deacetylase in the fungus or fungal unit thereof.

In certain embodiments of the sixth aspect, the method is an in vitro method for sensitizing a fungus or fungal unit thereof involved in infection and/or disease of keratinized tissue, such as onychomycosis, to an antifungal agent. If in vitro, inhibiting the activity of a histone deacetylase in the fungus or fungal unit thereof comprises contacting the fungus or fungal unit thereof with a sensitizing effective amount of an inhibitor of the activity of a histone deacetylase in the fungus or fungal unit thereof. In certain embodiments, the method is an in vivo method for sensitizing a fungus or fungal unit thereof involved in infection and/or disease of keratinized tissue, such as onychomycosis, to an antifungal compound. If in vivo, inhibiting the activity of a histone deacetylase in the fungus or fungal unit thereof comprises administering to a subject having growth of the fungus or fungal unit thereof thereon and/or therein, a sensitizing effective amount of an inhibitor of the activity of a histone deacetylase in the fungus or fungal unit thereof.

In certain embodiments of the sixth aspect, the inhibitor of the activity of a histone deacetylase in the fungus or fungal unit thereof may also be combined with an antifungal agent to form an antifungal mixture or a synergistic mixture thereof. Accordingly, in certain embodiments of the sixth aspect, the method further comprises contacting the fungus or fungal unit thereof with an antifungal agent, or, if in vivo, administering to the subject an antifungal agent. In certain embodiments of the sixth aspect, the inhibitor of the activity of a histone deacetylase in the fungus or fungal unit thereof and the antifungal agent are in respective proportions to provide a synergistic antifungal effect against the fungus or fungal unit thereof when compared to either the inhibitor alone or the antifungal agent alone. As will be understood by those skilled in the art, the synergistic effect may be obtained within various proportions of the inhibitor of the activity of a histone deacetylase in the fungus or fungal unit thereof and the antifungal agent, depending for example on the kind of fungus or fungal unit thereof towards which effect is measured. When used in combination with an antifungal agent(s), the inhibitor of the activity of a histone deacetylase in the fungus or fungal unit thereof can be formulated together with the antifungal agent(s), co-administered with the antifungal agent(s), or applied sequentially with the antifungal agent(s).

In accordance with certain embodiments of the sixth aspect, the present invention provides inhibitors of the activity of a histone deacetylase in a fungus or fungal unit thereof and synergistic combinations of said inhibitors and an antifungal agent, for sensitizing a fungus or fungal unit thereof involved in infection and/or disease of keratinized tissue, such as onychomycosis to the antifungal agent. In accordance with certain embodiments of the sixth aspect, the invention also provides for the use of an inhibitor of the activity of a histone deacetylase in a fungus or fungal unit thereof and a synergistic combination of said inhibitor and an antifungal agent, for sensitizing a fungus or fungal unit thereof involved in infection and/or disease of keratinized tissue, such as onychomycosis to the antifungal agent. Also in accordance with certain embodiments of the sixth aspect, the invention provides for the use of an inhibitor of the activity of a histone deacetylase in a fungus or fungal unit thereof or a synergistic combination of said inhibitor and an antifungal agent, in the manufacture of a medicament to sensitize a fungus or fungal unit thereof involved in infection and/or disease of keratinized tissue, such as onychomycosis, to the antifungal agent.

In certain embodiments of the sixth aspect, the sensitizing effect of the inhibitor of the activity of a histone deacetylase in the fungus or fungal unit thereof on the fungus or fungal unit thereof is greater than its effect on a human or other animal cell. In certain embodiments, the sensitizing effect is specific for the fungus or fungal unit thereof.

In a seventh aspect, the present invention provides a method for enhancing the activity of an antifungal agent against a fungus or fungal unit thereof involved in infection and/or disease of keratinized tissue, such as onychomycosis, comprising contacting the fungus or fungal unit thereof with the antifungal agent in combination with an activity enhancing effective amount of a compound according to the present invention.

In certain embodiments of the seventh aspect, the method is an in vitro method for enhancing the activity of an antifungal agent. In certain embodiments, the method is an in vivo method for enhancing the activity of an antifungal agent. If in vivo, the method comprises administering to a subject having growth of the fungus or fungal unit thereof thereon and/or therein, an activity enhancing effective amount of a compound according to the present invention.

In certain embodiments of the seventh aspect, the compounds according to the present invention may be combined with the antifungal agent to form an antifungal mixture or a synergistic mixture thereof. In certain embodiments of the seventh aspect, the compound according to the present invention and the antifungal agent are in respective proportions to provide a synergistic effect to enhance the activity of the antifungal agent when compared to the antifungal agent alone. As will be understood by those skilled in the art, the synergistic effect may be obtained within various proportions of the compound according to the present invention and the antifungal agent, depending for example on the kind of fungus or fungal unit thereof towards which effect is measured. When used in combination with the antifungal agent(s), the compounds according to the present invention can be formulated together with the other antifungal agent(s), co-administered with the other antifungal agent(s), or applied sequentially with the other antifungal agent(s).

In accordance with certain embodiments of the seventh aspect, the present invention provides compounds and synergistic combinations of said compounds and an antifungal agent, to enhance the activity of an antifungal agent against a fungus or fungal unit thereof involved in infection and/or disease of keratinized tissue, such as onychomycosis. In accordance with certain embodiments of the seventh aspect, the invention also provides for the use of a compound according to the present invention and a synergistic combination of said compound and an antifungal agent, for enhancing the activity of the antifungal agent against a fungus or fungal unit thereof involved in infection and/or disease of keratinized tissue, such as onychomycosis. Also in accordance with certain embodiments of the seventh aspect, the invention provides for the use of a compound according to the present invention or a synergistic combination of said compound and an antifungal agent, in the manufacture of a medicament to enhance the activity of the antifungal agent against a fungus or fungal unit thereof involved in infection and/or disease of keratinized tissue, such as onychomycosis.

In certain embodiments of the seventh aspect, the activity enhancing effect of the compound is more active against the fungus or fungal unit thereof than against a human or other animal cell. In certain embodiments, the activity enhancing effect of the compound is specific for the fungus or fungal unit thereof.

In an eighth aspect, the present invention provides a method for enhancing the activity of an antifungal agent against a fungus or fungal unit thereof involved in infection and/or disease of keratinized tissue, such as onychomycosis, comprising contacting the fungus or fungal unit thereof with the antifungal agent in combination with inhibiting the activity of a histone deacetylase in the fungus or fungal unit thereof.

In certain embodiments of the eighth aspect, the method is an in vitro method for enhancing the activity of an antifungal agent. If in vitro, inhibiting the activity of a histone deacetylase in the fungus or fungal unit thereof comprises contacting the fungus or fungal unit thereof with an activity inhibiting effective amount of an inhibitor of the activity of a histone deacetylase in the fungus or fungal unit thereof. In certain embodiments, the method is an in vivo method for enhancing the activity of an antifungal agent. If in vivo, contacting the fungus or fungal unit thereof with the antifungal agent in combination with inhibiting the activity of a histone deacetylase in the fungus or fungal unit thereof comprises administering to a subject having infection and/or disease of keratinized tissue, such as onychomycosis, thereon and/or therein, the antifungal agent in combination with administering to the subject an activity inhibiting effective amount of an inhibitor of the activity of a histone deacetylase in the fungus or fungal unit thereof.

In certain embodiments of the eighth aspect, the inhibitor of the activity of a histone deacetylase in the fungus or fungal unit thereof may be combined with the antifungal agent to form an antifungal mixture or a synergistic mixture thereof. In certain embodiments of the eighth aspect, the inhibitor of the activity of a histone deacetylase in the fungus or fungal unit thereof and the antifungal agent are in respective proportions to provide a synergistic effect to enhance the activity of the antifungal agent when compared to the antifungal agent alone. As will be understood by those skilled in the art, the synergistic effect may be obtained within various proportions of the inhibitor of the activity of a histone deacetylase in the fungus or fungal unit thereof and the antifungal agent, depending for example on the kind of fungus or fungal unit thereof towards which effect is measured. When used in combination the antifungal agent(s), the inhibitor of the activity of a histone deacetylase in the fungus or fungal unit thereof can be formulated together with the antifungal agent(s), co-administered with antifungal agent(s), or applied sequentially with the antifungal agent(s).

In accordance with certain embodiments of the eighth aspect, the present invention provides inhibitors of the activity of a histone deacetylase in a fungus or fungal unit thereof and synergistic combinations of said inhibitors and an antifungal agent, which enhance the activity of the antifungal agent against a fungus or fungal unit thereof involved in infection and/or disease of keratinized tissue, such as onychomycosis, compared to the activity of the antifungal agent alone. In accordance with certain embodiments of the eighth aspect, the invention also provides for the use of an Inhibitor of the activity of a histone deacetylase in a fungus or fungal unit thereof and a synergistic combination of said inhibitor and an antifungal agent, for enhancing the activity of an antifungal agent against a fungus or fungal unit thereof involved in infection and/or disease of keratinized tissue, such as onychomycosis. Also in accordance with certain embodiments of the eighth aspect, the invention provides for the use of an inhibitor of the activity of a histone deacetylase in a fungus or fungal unit thereof or a synergistic combination of said inhibitor and an antifungal agent, in the manufacture of a medicament to enhance the activity of the antifungal agent against a fungus or fungal unit thereof involved in infection and/or disease of keratinized tissue, such as onychomycosis.

In a ninth aspect, the present invention provides a kit, comprising a compound according to the present invention, and optionally instructions for using the kit in method according to the present invention.

In certain embodiments of the ninth aspect, the kit further comprises another antifungal agent. In certain embodiments, the compound and antifungal agent are mixed in respective proportions to provide a synergistic antifungal effect.

In a tenth aspect, the present invention provides a kit, comprising an inhibitor of fungal histone deacetylase, and optionally instructions for using the kit in a method according to the present invention.

In certain embodiments of the tenth aspect, the kit further comprises another antifungal agent. In certain embodiments, the inhibitor of fungal histone deacetylase and antifungal agent are mixed in respective proportions to provide a synergistic antifungal effect.

In accordance pith aspects of the present invention, certain embodiments relate to the methods mentioned above using the kits as described above.

The present invention also encompasses a product containing (a) a compound according to the present invention, or an N-oxide, hydrate, solvate, tautomer, pharmaceutically acceptable salt, prodrug or complex thereof, or a racemic or scalemic mixture, diastereomer or enantiomer thereof, as a first component, and (b) another antifungal agent as a second component, as a combination for use in a method or kit as described above, wherein said (a) and (b) are in respective proportions to provide a synergistic effect against the fungus or fungal unit.

The present invention also encompasses a product containing (a) an inhibitor of the activity of a histone deacetylase in the fungus or fungal unit thereof, as a first component, and (b) an antifungal agent as a second component, as a combination for use in a method or kit as described above, wherein said (a) and (b) are in respective proportions to provide a synergistic effect against the fungus or fungal unit.

In accordance with certain embodiments of aspects of the present invention, the compounds according to the present invention are compounds of Formula (I), Formula (Ia) or Formula (II) and N-oxides, hydrates, solvates, tautomers, pharmaceutically acceptable salts, prodrugs and complexes thereof, and racemic and scalemic mixtures, diastereomers and enantiomers thereof. In certain embodiments of aspects of the present invention, the compounds according to the present invention are prodrugs of Formula (Ib), or Formula (IIa) and N-oxides, hydrates, solvates, tautomers, pharmaceutically acceptable salts and complexes thereof, and racemic and scalemic mixtures, diastereomers and enantiomers thereof.

In certain embodiments of aspects of the present invention, the inhibitor of the activity of a histone deacetylase in the fungus or fungal unit thereof is an inhibitor of transcription or translation of a nucleic acid sequence encoding a product having histone deacetylase activity in the fungus or fungal unit thereof. In certain embodiments of aspects of the present invention, the inhibitor of transcription or translation is selected from the group consisting of antisense nucleic acid, short interfering RNA (siRNA), double-stranded RNA (dsRNA), micro-RNA (miRNA), and short hairpin RNA (shRNA). In certain embodiments of aspects of the present invention, the nucleic acid sequence comprises a gene selected from the group consisting of RPD3, HDA1, HOS1, HOS2, HOS3 and SIR2 and mutants, alleles and homologs thereof. In certain embodiments of aspects of the present invention, the nucleic acid sequence comprises the gene HOS2 or a mutant, allele or homolog thereof. In certain embodiments of aspects of the present invention, the nucleic acid sequence comprises the gene HOS2. In certain embodiments of aspects of the present invention, the inhibitor of the activity of a histone deacetylase in the fungus or fungal unit thereof is an inhibitor of enzymatic activity of a histone deacetylase in the fungus or fungal unit thereof. In certain embodiments of aspects of the present invention, the inhibitor of enzymatic activity is selected from the group consisting of an antibody or active fragment thereof and a small molecule. In certain embodiments of aspects of the present invention, the inhibitor of enzymatic activity is a small molecule. In certain embodiments of aspects of the present invention, the inhibitor of enzymatic activity is a hydroxamate-based small molecule.

In certain embodiments of aspects of the present invention, the inhibitor of the activity of a histone deacetylase in the fungus or fungal unit thereof is more active against a fungal histone deacetylase than a human or other animal histone deacetylase. In certain embodiments of aspects of the present invention the inhibitor is specific for one or more fungal histone deacetylase.

In certain embodiments of aspects of the present invention, the inhibitor of the activity of a histone deacetylase in the fungus or fungal unit thereof is a compound of Formula (I), Formula (Ia) or Formula (II) or an N-oxide, hydrate, solvate, tautomer, pharmaceutically acceptable salt, prodrug or complex thereof, or a racemic or scalemic mixture, diastereomer or enantiomer thereof. In certain embodiments of aspects of the present invention, the inhibitor of the activity of a histone deacetylase in the fungus or fungal unit thereof is a cleavage product of the prodrug of Formula (Ib) or Formula (IIa) or an N-oxide, hydrate, solvate, tautomer, pharmaceutically acceptable salt or complex thereof, or a racemic or scalemic mixture, diastereomer or enantiomer thereof.

In certain embodiments of aspects of the invention, the compounds according to the present invention are represented by the Formula (I):

Cy²-L²-Ar²—Y²—C(O)NH—Z (I)

and N-oxides, hydrates, solvates, tautomers, pharmaceutically acceptable salts, prodrugs and complexes thereof, and racemic and scalemic mixtures, diastereomers and enantiomers thereof, wherein
Cy² is H, cycloalkyl, aryl, heteroaryl, or heterocyclyl, any of which may be optionally substituted, provided that Cy² is not a (spirocycloalkyl)heterocyclyl;
L² is C₁-C₈ saturated alkylene or C₂-C₈ alkenylene, wherein the alkylene or alkenylene optionally may be substituted, and wherein one or two of the carbon atoms of the alkylene is optionally replaced by a heteroatomic moiety independently selected from the group consisting of O; NR′, R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z is selected from the group consisting of anilinyl, pyridyl, thiadiazolyl, each of which is optionally substituted, and —O-M, M being H or a pharmaceutically acceptable cation.

Substituted alkyl, alkenyl, cycloalkyl, aryl, heterocyclyl, and heteroaryl groups have one or more, for example between one and about three, alternatively one or two substituents, which are preferably selected from the group consisting of C₁-C₆ alkyl (for example, C₁-C₄ alkyl), halo (for example Cl, Br, or F), haloalkyl (for example, (halo)₁₋₅(C₁-C₆)alkyl, alternatively (halo)₁₋₅(C₁-C₃)alkyl, for example —CF₃), C₁-C₆ alkoxy (for example, methoxy, ethoxy, or benzyloxy), aryloxy (for example, phenoxy), C₁-C₆ alkoxycarbonyl (for example, C₁-C₃ alkoxycarbonyl, such as carbomethoxy or carboethoxy), C₆-C₁₀ aryl (for example, phenyl), (C₆-C₁₀)ar(C₁-C₆)alkyl (for example, (C₆-C₁₀)ar(C₁-C₃)alkyl, such as benzyl, naphthylmethyl or phenethyl), hydroxy(C₁-C₆)alkyl (for example, hydroxy(C₁-C₃)alkyl, such as hydroxymethyl), amino(C₁-C₆)alkyl (for example, amino(C₁-C₃)alkyl, such as aminomethyl), (C₁-C₆)alkylamino (for example, methylamino, ethylamino, or propylamino), di-(C₁-C₆)alkylamino (for example, dimethylamino or diethylamino), (C₁-C₆)alkylcarbamoyl (for example, methylcarbamoyl, dimethylcarbamoyl, or benzylcarbamoyl), (C₆-C₁₀)arylcarbamoyl (for example, phenylcarbamoyl), (C₁-C₆)alkaneacylamino (for example, acetylamino), (C₆-C₁₀)areneacylamino (for example, benzoylamino), (C₁-C₆)alkanesulthnyl (for example, methanesulfonyl), (C₁-C₆)alkanesulfonamido (for example, methanesulfonamido), (C₆-C₁₀)arenesulfonyl (for example, benzenesulfonyl or toluenesulfonyl), (C₆-C₁₀)arenesulfonamido (for example, benzenesulfonyl or toluenesulfonyl), (C₆-C₁₀)ar(C₁-C₆)alkylsulfonamido (for example, benzylsulfonamido), C₁-C₆ alkylcarbonyl (for example, C₁-C₃ alkylcarbonyl, acetyl), (C₁-C₆)acyloxy (for example, acetoxy), cyano, amino, carboxy, hydroxy, ureido and nitro. One or more carbon atoms of an alkyl, cycloalkyl, or heterocyclyl group may also be optionally substituted with an oxo group.

In certain embodiments of aspects of the invention, the compounds are represented by the Formula (I) wherein, when the carbon atom to which Cy² is attached is oxo substituted, then Cy² and Z are not both pyridyl.

In certain embodiments of aspects of the invention, the compounds are represented by the Formula (I) wherein, Cy² is C₆-C₁₄ aryl, for example C₆-C₁₀ aryl, which may be optionally substituted. In certain embodiments of the compounds of Formula (I), Cy² is phenyl or naphthyl, either of which may be optionally substituted. In certain embodiments of the compounds of Formula (I), Cy² is phenyl, which may be optionally substituted. In certain embodiments, Cy² is heteroaryl, which may be optionally substituted. In certain embodiments, Cy² is selected from the group consisting of pyridine, indole, thienyl, benzothienyl, furyl, benzofuryl, quinolyl, isoquinolyl, and thiazolyl, any of which may be optionally substituted. In certain embodiments, Cy² is substituted with one or more substituents independently selected from the group consisting of trihaloalkyl (for example, trifluoroalkyl), halogen, CN, C₁-C₆alkyl, amidine, sulfone, alkylsulfone, imidate and alkylimidate. In certain embodiments, Cy² is phenyl optionally substituted with one or more substituents independently selected from the group consisting of trihaloalkyl (for example trifluoroalkyl), halogen, CN, C₁-C₆alkyl, amidine, sulfone, alkylsulfone, imidate and alkylimidate; in certain embodiments, the substituents are independently selected from the group consisting of trihaloalkyl (for example trifluoroalkyl) and halogen. In certain embodiments, Cy² is unsubstituted phenyl.

In certain embodiments of aspects of the invention, the compounds are represented by the Formula (I) wherein, L² is C₁-C₈ saturated alkylene, wherein one of the carbon atoms of the saturated alkylene is replaced by a heteroatom moiety selected from the group consisting of O; NR′, R′ being alkyl, acyl, or hydrogen; S; S(O); or S(O)₂. In certain embodiments, the carbon atom adjacent to Cy² is replaced by a heteroatom moiety. In certain embodiments, L² is selected from the group consisting of —S—(CH₂)₂—, —S(O)—(CH₂)₂—, —S(O)₂—(CH₂)₂—, —S—(CH₂)₃—, —S(O)—(CH₂)₃—, and —S(O)₂—(CH₂)₃—. In certain embodiments, L² is selected from the group consisting of C₁-C₆ saturated alkylene, C₁-C₅ saturated alkylene, C₁-C₄ saturated alkylene, C₁-C₃ saturated alkylene, C₁-C₂ saturated alkylene and C₁ saturated alkylene, any of which groups may be optionally substituted. In certain embodiments, L² is selected from the group consisting of C₁-C₆ saturated alkylene, C₁-C₅ saturated alkylene, C₁-C₄ saturated alkylene and C₁-C₃ saturated alkylene, any of which groups may be optionally substituted. In certain embodiments. L² is C₁-C₄ saturated alkylene, which may be optionally substituted. In certain embodiments, L² is unsubstituted. In certain embodiments, L² is unsubstituted C₁-C₄ saturated alkylene. In certain embodiments, L² is selected from the group consisting of C₂-C₈ saturated alkylene, C₂-C₇ saturated alkylene, C₂-C₆ saturated alkylene, C₂-C₅ saturated alkylene, C₂-C₄ saturated alkylene and C₂-C₃ saturated alkylene, any of which groups may be optionally substituted. In certain embodiments, L² is substituted at one or two positions with a substituent independently selected from the group consisting of C₁-C₆ alkyl, C₆-C₁₀ aryl, amino, oxo, hydroxy, C₁-C₄ alkoxy, and C₆-C₁₀ aryloxy. In certain embodiments, the L² alkylene or alkenylene group is substituted with one or two of oxo or hydroxy.

In certain embodiments of aspects of the invention, the compounds are represented by the Formula (I) wherein, Ar² is C₆-C₁₄ arylene, for example C₆-C₁₀ arylene, any of which may be optionally substituted. In certain embodiments, Ar² is phenylene, for example 4-phenylene. In certain embodiments, the phenylene is fused to an aryl or heteroaryl ring, or to a saturated or partially unsaturated cycloalkyl or heterocyclic ring, any of which groups also may be optionally substituted.

In certain embodiments of aspects of the invention, the compounds are represented by the Formula (I) wherein, Y² is a chemical bond or is a straight- or branched-chain alkylene, which may be optionally substituted. In certain embodiments, Y² is a chemical bond, and the group —C(O)NH—Z is directly attached to Ar². In certain embodiments, Y² is alkylene; in certain embodiments saturated alkylene. In certain embodiments, the saturated alkylene is C₁-C₈ alkylene. In certain embodiments, Y² is C₁-C₆ alkylene, alternatively C₁-C₃ alkylene, alternatively C₁-C₂ alkylene, any of which may be optionally substituted. In certain embodiments, Y² is methylene.

In certain embodiments of aspects of the invention, the compounds are represented by the Formula (I) wherein, Z is —OH.

In certain embodiments of aspects of the invention, the compounds are represented by the Formula (Ia):

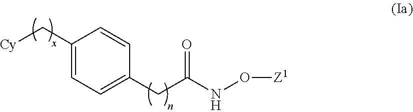

N-oxides, hydrates, solvates, tautomers, pharmaceutically acceptable salts, prodrugs and complexes thereof, and racemic and scalemic mixtures, diastereomers and enantiomers thereof, wherein
Cy is alkyl, cycloalkyl, aryl, heteroaryl or heterocyclyl, any of which maybe optionally substituted;
x is an integer from 0 to 5, wherein the chain of length x is optionally substituted and wherein one or two carbon atoms of the chain of length x is optionally replaced with a heteroatom;
n is an integer from 0 to 2; and
Z¹ is selected from the group consisting of H and a heterocyclic group;
with the provisos that when x is 4, n is not 2, and when x is 3, n is not 3.

In certain embodiments of aspects of the invention, the compounds are represented by the Formula (Ia) wherein, Cy is cycloalkyl, aryl, heteroaryl or heterocyclyl, any of which may be optionally substituted.

In certain embodiments of aspects of the invention, the compounds are represented by the Formula (Ia) wherein, Cy optionally has one or more, for example between one and about three, alternatively one or two substituents, which are selected from the group consisting of C₁-C₆ alkyl (for example, C₁-C₄ alkyl), halo (for example Cl, Br, or F), haloalkyl (for example, (halo)₁₋₅(C₁-C₆)alkyl, alternatively (halo)₁₋₅(C₁-C₃)alkyl, for example CF₃), C₁-C₆ alkoxy (for example, methoxy, ethoxy, or benzyloxy), C₆-C₁₀ aryloxy (for example, phenoxy), C₁-C₆ alkoxycarbonyl (for example, C₁-C₃ alkoxycarbonyl, such as carbomethoxy or carboethoxy), C₆-C₁₀ aryl (for example, phenyl), (C₆-C₁₀)ar(C₁-C₆)alkyl (for example, (C₆-C₁₀)ar(C₁-C₃)alkyl, such as benzyl, naphthylmethyl or phenethyl), hydroxy(C₁-C₆)alkyl (for example, hydroxy(C₁-C₃)alkyl, such as hydroxymethyl), amino(C₁-C₆)alkyl (for example, amino(C₁-C₃)alkyl, such as aminomethyl), (C₁-C₆)alkylamino (for example, methylamino, ethylamino, or propylamino), di-(C₁-C₆)alkylamino (for example, dimethylamino or diethylamino), (C₁-C₆)alkylcarbamoyl (for example, methylcarbamoyl, dimethylcarbamoyl, or benzylcarbamoyl), (C₆-C₁₀)arylcarbamoyl (for example, phenylcarbamoyl), (C₁-C₆)alkaneacylamino (for example, acetylamino), (C₆-C₁₀)areneacylamino (for example, benzoylamino), (C₁-C₆)alkanesulfonyl (for example, methanesulfonyl), (C₁-C₆)alkanesulfonamido (for example, methanesulfonamido), (C₆-C₁₀)arenesulfonyl (for example, benzenesulfonyl or toluenesulfonyl), (C₆-C₁₀)arenesulfonamido (for example, benzenesulfonyl or toluenesulfonyl), (C₆-C₁₀)ar(C₁-C₆)alkylsulfonamido (for example, benzylsulfonamido), C₁-C₆ alkylcarbonyl (for example, C₁-C₃ alkylcarbonyl, acetyl), (C₁-C₆)acyloxy (for example, acetoxy), cyano, amino, carboxy, hydroxy, ureido and nitro. In certain embodiments, one or more carbon atoms of Cy may also be optionally substituted with an oxo group.

In certain embodiments of aspects of the invention, the compounds are represented by the Formula (Ia) wherein, Cy is unsubstituted or is substituted by one or two substituents independently selected from the group consisting of C₁-C₄ alkyl, C₁-C₄ haloalkyl, C₆-C₁₀ aryl, (C₆-C₁₀)ar(C₁-C₆)alkyl, halo, nitro, hydroxy, C₁-C₆ alkoxy, C₁-C₆ alkoxycarbonyl, carboxy, and amino.

In certain embodiments of aspects of the invention, the compounds are represented by the Formula (Ia) wherein, Cy is phenyl, pyridine or indole, for example phenyl or indole. In certain embodiments, Cy is phenyl.

In certain embodiments of aspects of the invention, the compounds are represented by the Formula (Ia) wherein, Cy is optionally substituted with one or more substituents independently selected from the group consisting of alkyl, alkenyl, alkynyl, trihaloalkyl, halogen, CN, amidine, alkylamidine, sulfone, alkylsulfone, imidate and alkylimidate.

In certain embodiments of aspects of the invention, the compounds are represented by the Formula (Ia) wherein, Cy is phenyl or indole, optionally substituted with one or more substituents independently selected from the group consisting of alkyl, alkenyl, alkynyl, trihaloalkyl, halogen, CN, amidine, alkylamidine, sulfone, alkylsulfone, imidate and alkylimidate, alternatively one or more substituents independently selected from the group consisting of alkyl, alkenyl, alkynyl, trihaloalkyl and halogen.

In certain embodiments of aspects of the invention, the compounds are represented by the Formula (Ia) wherein, x is an integer from 2 to 4, alternatively 3 to 4. In certain embodiments, n is an integer from 1 to 2, for example, 1.

In certain embodiments of aspects of the invention, the compounds are represented by the Formula (Ia) wherein, Z¹ is H.

In certain embodiments of aspects of the invention, the compounds are represented by the Formula (Ia) wherein, one carbon atom of the chain of length x is replaced with a heteroatom, for example, S.

In certain embodiments of aspects of the invention, the compound is selected from the group consisting of

| Compound No. | |
|---|---|
| 1 | 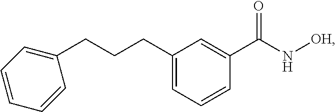 |
| 2 | 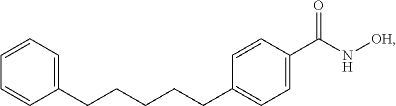 |
| 3 | 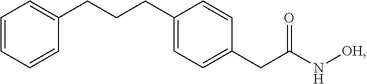 |
| 4 | 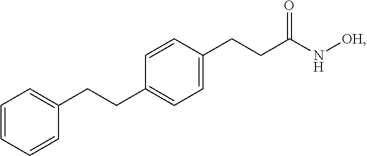 |
| 5 | 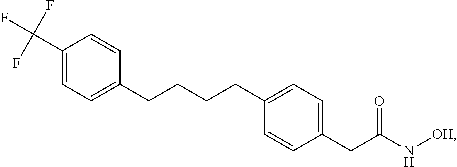 |
| 6 | 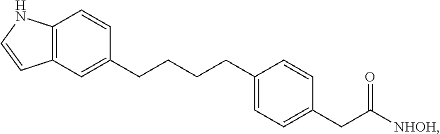 |
| 7 | 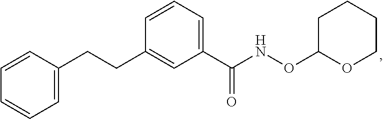 |
| 8 | 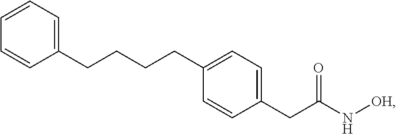 |
| 9 | 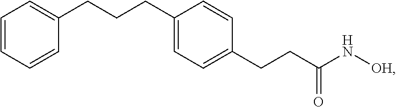 |
| 10 | 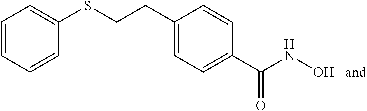 |
| 11 | 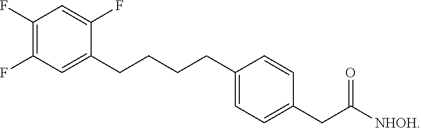 |

and N-oxides, hydrates, solvates, tautomers, pharmaceutically acceptable salts, prodrugs and complexes thereof, and racemic and scalemic mixtures, diastereomers and enantiomers thereof.

In certain embodiments of aspects of the invention, the compounds are selected from the group consisting of

| Compound No. | |
|---|---|
| 3 | 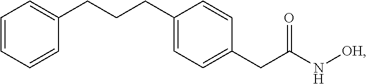 |
| 5 | 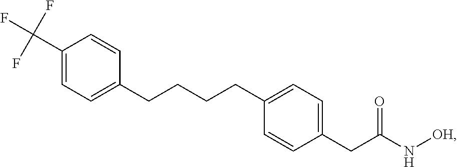 |
| 6 | 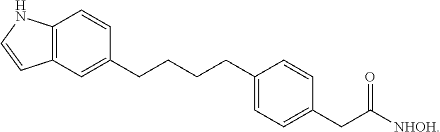 |
| 8 | 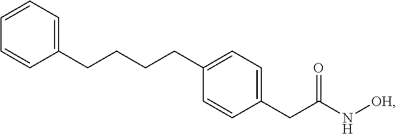 |
| 11 | 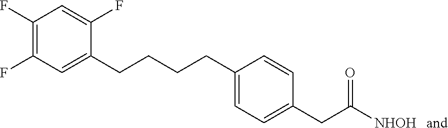 |

and N-oxides, hydrates, solvates, tautomers, pharmaceutically acceptable salts, prodrugs and complexes thereof, and racemic and scalemic mixtures, diastereomers and enantiomers thereof.

In certain embodiments of aspects of the present invention, the compound is

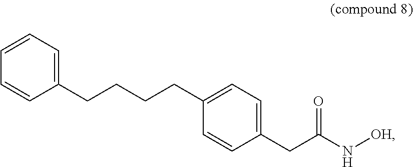

or a hydrate, solvate, tautomer, pharmaceutically acceptable salt, prodrug or complex thereof, and racemic and scalemic mixtures, diastereomers and enantiomers thereof.

In certain embodiments of aspects of the invention, the compounds are represented by a prodrug of Formula (Ib):

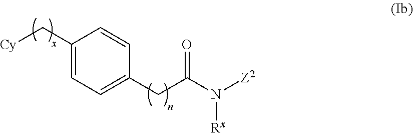

and N-oxides, hydrates, solvates, tautomers, pharmaceutically acceptable salts and complexes thereof, and racemic and scalemic mixtures, diastereomers and enantiomers thereof,

wherein
Cy is as defined for formula (Ia);
x is an integer from 0 to 5, wherein the chain of length x is optionally substituted and wherein one or two carbon atoms of the chain of length x is optionally replaced with a heteroatom;
n is an integer from 0 to 2;
R^{x} is H or —OH;
Z² is —R²⁰, —O—R²⁰, —R²¹, or

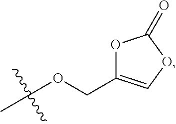

wherein —R²⁰ is selected from the group consisting of —C(O)—R¹⁰, —C(O)O—R¹⁰, —R¹¹, —CH(R¹²)—O—C(O)—R¹⁰, —C(O)—[C(R¹⁰)(R^{10′})]₁₋₄—NH(R¹³), —S(O₂) R¹⁰, —P(O)(OR¹⁰)(OR¹⁰), —C(O)—(CH₂)ₙ—CH(OH)—CH₂—O—R¹⁰, —C(O)—O—(CH₂)ₙ—CH(OH)—CH₂—O—R¹⁰ and —C(O)—(CH₂)ₙ—C(O)OR¹⁰, provided that the N to which Z is bound is not directly bound to two oxygen atoms; or
R^{x} is absent and R²⁰ forms an optionally substituted heterocyclic ring with the N to which it is attached;
n is 1-4;
R¹⁰ is selected from the group consisting of hydrogen, optionally substituted C₁-C₂₀ alkyl, optionally substituted C₂-C₂₀ alkenyl, optionally substituted C₂-C₂₀ alkynyl, optionally substituted C₁-C₂₀ alkoxycarbonyl, optionally substituted cycloalkyl, optionally substituted heterocycloalkyl, optionally substituted aryl, optionally substituted heteroaryl, optionally substituted cycloalkylalkyl, optionally substituted heterocycloalkylalkyl, optionally substituted arylalkyl, optionally substituted heteroarylalkyl, optionally substituted cycloalkylalkenyl, optionally substituted heterocycloalkylalkenyl, optionally substituted arylalkenyl, optionally substituted heteroarylalkenyl, optionally substituted cycloalkylalkynyl, optionally substituted heterocycloalkylalkynyl, optionally substituted arylalkynyl, optionally substituted heteroarylalkynyl, a sugar residue and an amino acid residue (preferably bonded through the carboxy terminus of the amino acid);
R^{10′} is hydrogen, or
R¹⁰ and R^{10′} together with the carbon atom to which they are attached form an optionally substituted spirocycloalkyl;
R²¹ is—amino acid—R¹³, wherein R¹³ is covalently bound to the N-terminus;
R¹¹ is selected from the group consisting of hydrogen, optionally substituted alkyl, optionally substituted cycloalkyl, optionally substituted heterocycloalkyl, optionally substituted aryl, and optionally substituted heteroaryl;
R¹² is selected from hydrogen or alkyl; and
R¹³ is selected from the group consisting of hydrogen, an amino protecting group and R¹⁰.

with the provisos that when x is 4, n is not 2, and when x is 3, n is not 3.

In certain embodiments of aspects of the invention, the compounds are represented by the Formula (Ib) wherein, Z² is —O—C(O)—R¹⁰, —O—C(O)—[C(R¹⁰)(R^{10′})]₁₋₄—NH(R¹³) or —OR¹¹.

In certain embodiments of aspects of the invention, the compounds are represented by the Formula (Ib) wherein, the group R¹⁰ is an amino acid, wherein the amino acid is an L-amino acid.

In certain embodiments of aspects of the invention, the compounds are represented by the Formula (Ib) wherein, the group R¹⁰ is a sugar residue, wherein the sugar residue is a saccharide selected from the group consisting of glucose, galactose, mannose, gulose, idose, talose, allose, altrose, fructose, rhamnose, ribose and xylose.

In certain embodiments of aspects of the invention, the compound is a prodrug selected from the group consisting of

| Compound No. | |
|---|---|
| 12 | 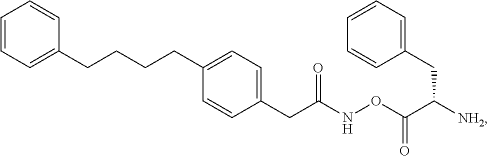 |
| 13 | 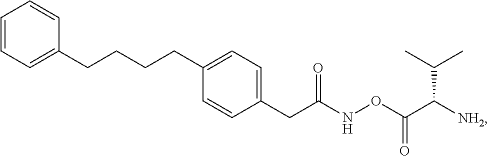 |
| 14 | 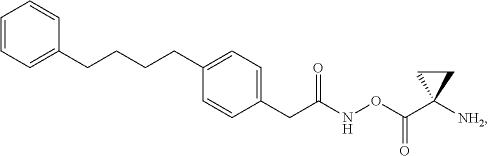 |
| 15 | 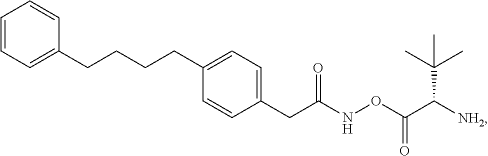 |
| 16 | 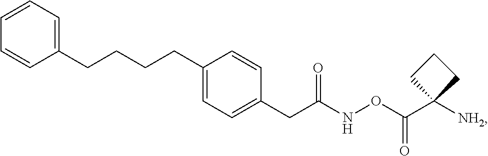 |
| 17 | 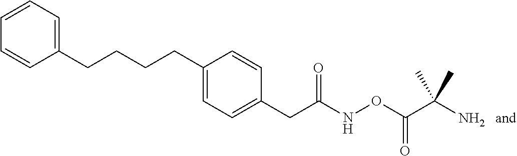 |
| 18 | 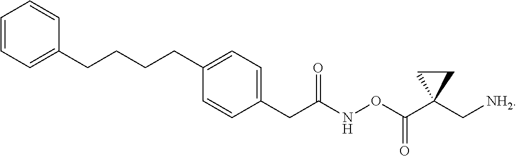 |

In certain embodiments of aspects of the invention, the prodrug is

| Compound No. | |
|---|---|
| 14 | 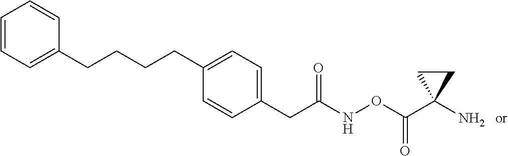 |
| 18 | 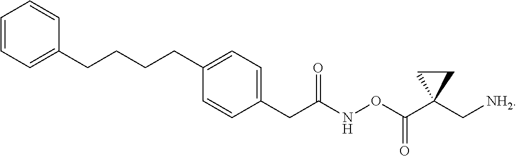 |

In certain embodiments of aspects of the invention, the compounds are represented by the Formula (II):

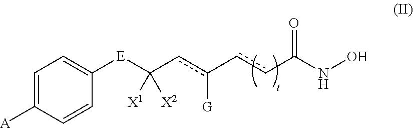

and N-oxides, hydrates, solvates, tautomers, pharmaceutically acceptable salts, prodrugs and complexes thereof, and racemic and scalemic mixtures, diastereomers and enantiomers thereof, wherein
A is selected from the group consisting of —O(CH₃), —NH₂ and aryl, wherein the aryl is optionally connected to the phenyl via a covalent bond or the aryl is fused to the phenyl;
E is selected from the group consisting of CH₂, CH(OCH₃), C═N(OH), C═CH₂ and O;
X¹ and X² are independently selected from the group consisting of H and CH₃;
G is selected from the group consisting of H and CH₃;

[symbol] is selected from the group consisting of a single bond and a double bond; and
t is an integer from 0 to 1,
with the proviso that the compound of formula (II) is not a compound selected from the group consisting of

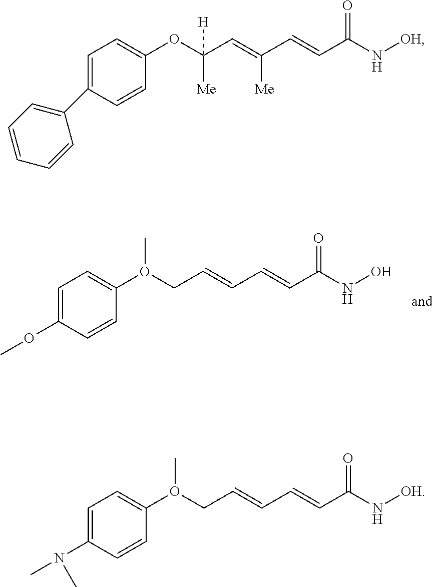

In certain embodiments of aspects of the present invention, the compounds are represented by a prodrug of Formula (IIa):

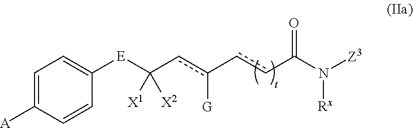

and N-oxides, hydrates, solvates, tautomers, pharmaceutically acceptable salts and complexes thereof, and racemic and scalemic mixtures, diastereomers and enantiomers thereof wherein
A, E, X¹, X², G and t are defined in Formula (II);
R^{x} is H or —OH; and
Z³ is —R²⁰, —O—R²⁰, —R²¹, or

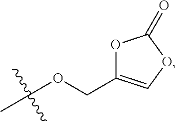

wherein —R²⁰ is selected from the group consisting of —C(O)—R¹⁰, —C(O)O—R¹⁰, —R¹¹, —CH(R¹²)—O—C(O)—R¹⁰, —C(O)—[C(R¹⁰)(R^{10′})]₁₋₄—NH(R¹³), —S(O₂) R¹⁰, —P(O)(OR¹⁰)(OR¹⁰), —C(O)—(CH₂)ₙ—CH(OH)—CH₂—O—R¹⁰, —C(O)—O—(CH₂)ₙ—CH(OH)—CH₂—O—R¹⁰ and —C(O)—(CH₂)ₙ—C(O)OR¹⁰, provided that the N to which Z is bound is not directly bound to two oxygen atoms; or
R^{x} is absent and R²⁰ forms an optionally substituted heterocyclic ring with the N to which it is attached;
n is 1-4;
R¹⁰ is selected from the group consisting of hydrogen, optionally substituted C₁-C₂₀ alkyl, optionally substituted C₂-C₂₀ alkenyl, optionally substituted C₂-C₂₀ alkynyl, optionally substituted C₁-C₂₀ alkoxycarbonyl, optionally substituted cycloalkyl, optionally substituted heterocycloalkyl, optionally substituted aryl, optionally substituted heteroaryl, optionally substituted cycloalkylalkyl, optionally substituted heterocycloalkylalkyl, optionally substituted arylalkyl, optionally substituted heteroarylalkyl, optionally substituted cycloalkylalkenyl, optionally substituted heterocycloalkylalkenyl, optionally substituted arylalkenyl, optionally substituted heteroarylalkenyl, optionally substituted cycloalkylalkynyl, optionally substituted heterocycloalkylalkynyl, optionally substituted arylalkynyl, optionally substituted heteroarylalkynyl, a sugar residue and an amino acid residue (preferably bonded through the carboxy terminus of the amino acid);
R^{10′} is hydrogen, or
R¹⁰ and R^{10′} together with the carbon atom to which they are attached form an optionally substituted spirocycloalkyl;
R²¹ is—amino acid—R¹³, wherein R¹³ is covalently bound to the N-terminus;
R¹¹ is selected from the group consisting of hydrogen, optionally substituted alkyl, optionally substituted cycloalkyl, optionally substituted heterocycloalkyl, optionally substituted aryl, and optionally substituted heteroaryl;
R¹² is selected from hydrogen or alkyl; and
R¹³ is selected from the group consisting of hydrogen, an amino protecting group and R¹⁰.

In certain embodiments of aspects of the invention, the compounds are represented by a prodrug of Formula (IL) wherein, Z³ is —O—C(O)—R¹⁰, —O—C(O)—[C(R¹⁰)(R¹⁰′)]₁₋₄—NH(R¹³) or —OR¹¹.

In certain embodiments of aspects of the invention, the compounds are represented by a prodrug of Formula (IIa) wherein, the group R¹⁰ is an amino acid, wherein the amino acid is an L-amino acid.

In certain embodiments of aspects of the invention, the compounds are represented by a prodrug of Formula (IIa) wherein, the group R¹⁰ is a sugar residue, wherein the sugar residue is a saccharide selected from the group consisting of glucose, galactose, mannose, gulose, idose, talose, allose, altrose, fructose, rhamnose, ribose and xylose.

In certain embodiments of aspects of the invention, the compounds are represented by a prodrug of Formula (IIa) wherein, A is NH₂.

In certain embodiments of aspects of the invention, the compounds are represented by a prodrug of Formula (IIa) wherein, A is aryl, preferably phenyl.

In certain embodiments of aspects of the invention, the compounds are represented by a prodrug of Formula (IIa) wherein, E is CH₂ or C═N(OH).

In certain embodiments of aspects of the invention, the compounds are represented by a prodrug of Formula (IIa) wherein, one of X¹ and X² is CH₃.

In certain embodiments of aspects of the invention, the compounds are represented by a prodrug of Formula (IIa) wherein, Z³ is CH₃.

In certain embodiments of aspects of the invention, the compounds are represented by a prodrug of Formula (IIa) wherein, [symbol] is a double bond.

In certain embodiments of aspects of the invention, the compounds are represented by a prodrug of Formula (IIa) wherein, t is 0.

In certain embodiments of aspects of the invention, the compounds are represented by a compound of Formula (II) wherein the compound is selected from the group consisting of

| Compound No. | |
|---|---|
| 19 | 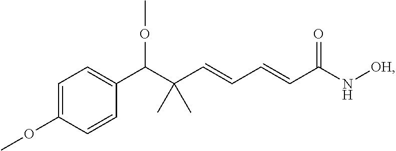 |
| 20 | 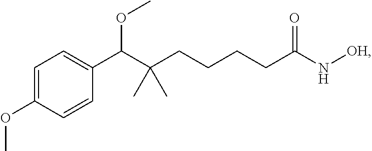 |
| 21 | 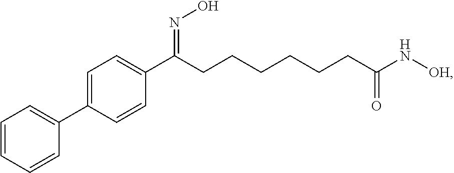 |
| 22 | 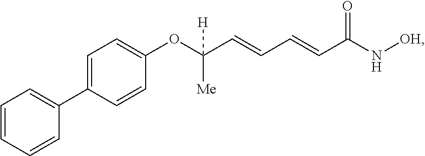 |
| 23 | 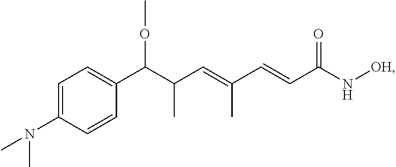 |
| 24 | 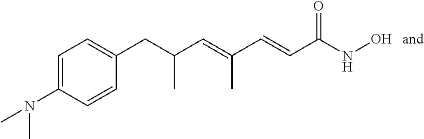 |
| 25 | 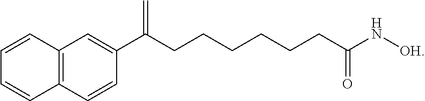 |

and N-oxides, hydrates, solvates, tautomers, pharmaceutically acceptable salts, prodrugs and complexes thereof, and racemic and scalemic mixtures, diastereomers and enantiomers thereof.

In certain embodiments of aspects of the present invention, the compound is

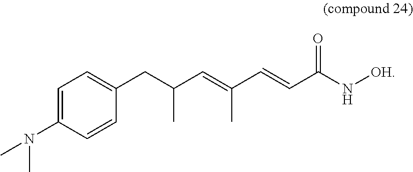

or an N-oxide, hydrate, solvate, tautomer, pharmaceutically acceptable salt, prodrug or complex thereof, and racemic and scalemic mixtures, diastereomers and enantiomers thereof.

Naturally-occurring or non-naturally occurring amino acids are used to prepare the prodrugs of the invention. In particular, standard amino acids suitable as a prodrug moiety include valine, leucine, isoleucine, methionine, phenylalanine, asparagine, glutamic acid, glutamine, histidine, lysine, arginine, aspartic acid, glycine, alanine, serine, threonine, tyrosine, tryptophan, cysteine and proline. Optionally an included amino acid is an α-, β-, or γ-amino acid. Also, naturally-occurring, non-standard amino acids can be utilized in the compositions and methods of the invention. For example, in addition to the standard naturally occurring amino acids commonly found in proteins, naturally occurring amino acids also illustratively include 4-hydroxyproline, .gamma.-carboxyglutamic acid, selenocysteine, desmosine, 6-N-methyllysine, .epsilon.-N,N,N-trimethyllysine, 3-methylhistidine, O-phosphoserine, 5-hydroxylysine, .epsilon.-N-acetyllysine, .omega.-N-methylarginine, N-acetylserine, .gamma.-aminobutyric acid, citrulline, ornithine, azaserine, homocysteine, .beta.-cyanoalanine and S-adenosylmethionine. Non-naturally occurring amino acids include phenyl glycine, meta-tyrosine, para-amino phenylalanine, 3-(3-pyridyl)-L-alanine-, 4-(trifluoromethyl)-D-phenylalanine, and the like.

In certain embodiments of aspects of the invention, the compounds comprise those of Formula (Ib) and Formula (IIa) as defined above, except that R²⁰ of Z² (of Formula Ia), and Z³ (of Formula IIa) is described in U.S. Pat. No. 4,443,435 (incorporated by reference in its entirety) as comprising —CH(R¹³⁰)—X¹⁵—C(O)—R¹³¹ wherein
X¹⁵ is O, S, or NR¹³²;
R¹³¹ is
(a) straight or branched chain alkyl having from 1 to 20 carbon atoms especially methyl, ethyl, isopropyl, t-butyl, pentyl or hexyl;
(b) aryl having from 6 to 10 carbon atoms especially phenyl, substituted phenyl or naphthalene;
(c) cycloalkyl having from 3 to 8 carbon atoms especially cyclopentyl, or cyclohexyl;
(d) alkenyl having from 2-20 carbon atoms especially C₂₋₆ alkenyl such as vinyl, allyl, or butenyl;
(e) cycloalkenyl having from 5 to 8 carbon atoms especially cyclopentenyl or cyclohexenyl;
(f) alkynyl having from 2 to 20 carbon atoms especially C₂₋₆ alkynyl for example, ethynyl, propynyl or hexynyl;
(g) aralkyl, alkaryl, aralkenyl, aralkynyl, alkenylaryl or alkynylaryl wherein alkyl, aryl, alkenyl and alkynyl are as previously defined;
(h) lower alkoxycarbonyl especially C₁-C₆ alkoxycarbonyl such as methoxycarbonyl, ethoxycarbonyl, t-butoxycarbonyl and cyclopentoxycarbonyl;
(i) carboxyalkyl or alkanoyloxyalkyl especially carboxy-C₁₋₆ alkyl such as formyloxymethyl and formyloxypropyl; or C₁₋₆ (alkylcarboxyalkyl) such as acetoxymethyl, n-propanoyloxyethyl and pentanoyloxybutyl;
(j) saturated or unsaturated monoheterocyclic or polyheterocyclic, or fused heterocyclic, either directly bonded to the carbonyl function or linked thereto via an alkylene bridge, containing from 1 to 3 of any one or more of the heteroatoms N, S or O in each heterocyclic ring thereof and each such ring being from 3- to 8-membered; and
(k) mono- or polysubstituted derivatives of the above, each of said substituents being selected from the group consisting of lower alkyl; lower alkoxy; lower alkanoyl; lower alkanoyloxy; halo especially bromo, chloro, or fluoro; haloloweralkyl especially fluoro, chloro or bromoloweralkyl such as trifluoromethyl and 1-chloropropyl; cyano; carbethoxy; loweralkylthio, especially C1-6 loweralkylthio such as methylthio, ethylthio and n-propylthio; nitro; carboxyl; amino; loweralkylamino especially C1-6 alkylamino, for example, methylamino, ethylamino and n-butylamino; diloweralkylamino especially di(C1-6 loweralkyl)amino such as N,N-dimethylamino, N,N-diethylamino and N,N-dihexylamino; carbamyl; loweralkylcarbamyl especially C1-6 alkylcarbamyl such as methylcarbamyl and ethyl carbamoyl; and
(l) R¹³³—X—C(O)-phenyl-, wherein R¹³³ is hydrogen or alkyl having from 1 to 10 carbons;
R¹³⁰ is hydrogen, (b) R¹³¹, lower alkanoyl, cyano, haloloweralkyl, carbamyl, loweralkylcarbamyl, or diloweralkylcarbamyl, —CH₂ONO₂, or —CH₂OCOR¹³¹; wherein
R¹³² is hydrogen or lower alkyl;
and further wherein R¹³¹ and R¹³⁰ may be taken together to form a ring cyclizing moiety selected from the group consisting of:

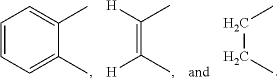

In certain embodiments of aspects of the invention, the compounds comprise those of Formula (Ib) and Formula (IIa) as defined above, except that R²⁰ of Z² (of Formula Ib) and Z³ (of Formula IIa) is described in U.S. Pat. No. 6,407,235 (incorporated by reference in its entirety) as comprising:
a) —C(O)(CH₂)ₘC(O)OR⁴⁰, wherein m is 1, 2, 3 or 4;
b)

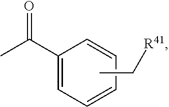

wherein R⁴¹ is —N(R⁴²)(R⁴³)and R⁴² and R⁴³ are hydrogen or lower alkyl, or is a five or six member heterocyclyl or heteroaryl optionally substituted by lower alkyl, or
c) —C(O)(CH₂)NHC(O)(CH₂)N(R⁴²)(R⁴³).

In certain embodiments of aspects of the invention, the compounds comprise those of Formula (Ib) and Formula (IIa) as defined above, except that R²⁰ of Z² (of Formula Ib) and Z³ (of Formula IIa) is described in U.S. Pat. No. 6,545,131 (incorporated by reference in its entirety) as comprising:

CO—(CH═CH)ₙ₁—(CH₂)ₙ₂—Ar—NH₂, —CO—(CH₂)ₙ₂—(CH═CH)ₙ₁—Ar—NR₂, CO—(CH₂)ₙ₂—(CH═CH)ₙ₁—CO—NH—Ar—NH₂ and CO—(CH═CH)ₙ₁—(CH₂)ₙ₂—CO—NH—Ar—NH₂ and substituted variations thereof, where n1 and n2 are from 0 to 5, Ar is a substituted or unsubstituted aryl group. In certain embodiments, Z² or Z³ is CO—(CH₂)ₙ₃—NH₂, where n3 is from 0 to 15, alternatively 3-15, and alternatively 6-12. In certain embodiments, substituent groups within this class are 6-aminohexanoyl, 7-aminoheptanoyl, 8-aminooctanoyl, 9-aminononanoyl, 10-aminodecanoyl, 11-aminoundecanoyl, and 12-aminododecanoyl. These substituents are generally synthesized from the corresponding amino acids, 6-aminohexanoic acid, and so forth. The amino acids are N-terminal protected by standard methods, for example Boc protection. Dicyclohexylcarbodiimide (DCCl)-promoted coupling of the N-terminal protected substituent to thapsigargin, followed by standard deprotection reactions produces primary amine-containing thapsigargin analogs.

In certain embodiments of aspects of the invention, the compounds comprise those of Formula (Ib) and Formula (IIa) as defined above, except that R²⁰ of Z² (of Formula Ib) and Z³ (of Formula IIa) is described in U.S. Pat. No. 7,115,573 (incorporated by reference in its entirety) as comprising:
an oligopeptide of the formula (AA)ₙ-AA³-AA²-AA¹, wherein: each AA independently represents an amino acid, n is 0 or 1, and when n is 1, then (AA)ₙ is AA⁴ which represents any amino acid, AA³ represents isoleucine, AA² represents any amino acid, and AA¹ represents any amino acid,
(2) a stabilizing group, and
(3) optionally, a linker group not cleavable by a trouase, such as TOP (described in greater detail below)
wherein the oligopeptide is directly linked to the stabilizing group at a first attachment site of the oligopeptide and the oligopeptide is directly linked to the therapeutic agent or indirectly linked through the linker group to the therapeutic agent at a second attachment site of the oligopeptide,
wherein the stabilizing group hinders cleavage of the compound by enzymes present in whole blood, and
wherein the compound is cleavable by an enzyme associated with the target cell, the enzyme associated with the target cell being other than TOP (Thimet oligopeptidase). The compound preferably includes an oligopeptide that is resistant to cleavage by a trouase, particularly TOP, i.e., resistant to cleavage under physiological conditions. The optionally present linker group that is not cleavable by a trouase is not cleavable under physiological conditions.

The typical orientation of these portions of the prodrug is as follows: (stabilizing group)-(oligopeptide)-(optional linker group)-(therapeutic agent).

Direct linkage of two portions of the prodrug means a covalent bond exists between the two portions. The stabilizing group and the oligopeptide are therefore directly linked via a covalent chemical bond at the first attachment site of the oligopeptide, typically the N-terminus of the oligopeptide. When the oligopeptide and the therapeutic agent are directly linked then they are covalently bound to one another at the second attachment site of the oligopeptide. The second attachment site of the oligopeptide is typically the C-terminus of the oligopeptide, but may be elsewhere on the oligopeptide.

Indirect linkage of two portions of the prodrug means each of the two portions is covalently bound to a linker group. In an alternative embodiment, the prodrug has indirect linkage of the oligopeptide to the therapeutic agent. Thus, typically, the oligopeptide is covalently bound to the linker group which, in turn, is covalently bound to the therapeutic agent.

In an alternative embodiment, the orientation of the prodrug may be reversed so that a stabilizing group is attached to the oligopeptide at the C-terminus and the therapeutic agent is directly or indirectly linked to the N-terminus of the oligopeptide. Thus, in an alternative embodiment, the first attachment site of the oligopeptide may be the C-terminus of the oligopeptide and the second attachment site by the oligopeptide may be the N-terminus of the oligopeptide. The linker group may optimally be present between the therapeutic agent and the oligopeptide. The alternative embodiment of the prodrug of the invention functions in the same manner as does the primary embodiment.

The stabilizing group typically protects the prodrug from cleavage by proteinases and peptidases present in blood, blood serum, and normal tissue. Particularly, since the stabilizing group caps the N-terminus of the oligopeptide, and is therefore sometimes referred to as an N-cap or N-block, it serves to ward against peptidases to which the prodrug may otherwise be susceptible. A stabilizing group that hinders cleavage of the oligopeptide by enzymes present in whole blood is chosen from the following:
(1) other than an amino acid, and
(2) an amino acid that is either (i) a non-genetically-encoded amino acid or (ii) aspartic acid or glutamic acid attached to the N-terminus of the oligopeptide at the β-carboxyl group of aspartic acid or the γ-carboxyl group of glutamic acid.

For example, dicarboxylic (or a higher order carboxylic) acid or a pharmaceutically acceptable salt thereof may be used as a stabilizing group. Since chemical radicals having more than two carboxylic acids are also acceptable as part of the prodrug, the end group having dicarboxylic (or higher order carboxylic) acids is an exemplary N-cap. The N-cap may thus be a monoamide derivative of a chemical radical containing two or more carboxylic acids where the amide is attached onto the amino terminus of the peptide and the remaining carboxylic acids are free and uncoupled. For this purpose, the N-cap is preferably succinic acid, adipic acid, glutaric acid, or phthalic acid, with succinic acid and adipic acid being most preferred. Other examples of useful N-caps in the prodrug compound of the invention include diglycolic acid, fumaric acid, naphthalene dicarboxylic acid, pyroglutamic acid, acetic acid, 1- or 2-, naphthylcarboxylic acid, 1,8-naphthyl dicarboxylic acid, aconitic acid, carboxycinnamic acid, triazole dicarboxylic acid, gluconic acid, 4-carboxyphenyl boronic acid, a (PEG).sub.n-analog such as polyethylene glycolic acid, butane disulfonic acid, maleic acid, nipecotic acid, and isonipecotic acid.

Further, a non-genetically encoded amino acid such as one of the following may also be used as the stabilizing group: β-Alanine, Thiazolidine-4-carboxylic acid, 2-Thienylalanine, 2-Naphthylalanine, D-Alanine, D-Leucine, D-Methionine, D-Phenylalanine, 3-Amino-3-phenylpropionic acid, γ-Aminobutyric acid, 3-amino-4,4-diphenylbutyric acid, Tetrahydroisoquinoline-3-carboxylic acid, 4-Aminomethylbenzoic acid, and Aminoisobutyric acid.

A linker group between the oligopeptide and the therapeutic agent may be advantageous for reasons such as the following: 1. As a spacer for steric considerations in order to facilitate enzymatic release of the AA¹ amino acid or other enzymatic activation steps. 2. To provide an appropriate attachment chemistry between the therapeutic agent and the oligopeptide. 3. To improve the synthetic process of making the prodrug conjugate (e.g., by pre-derivitizing the therapeutic agent or oligopeptide with the linker group before conjugation to enhance yield or specificity.) 4. To improve physical properties of the prodrug. 5. To provide an additional mechanism for intracellular release of the drug.

Linker structures are dictated by the required functionality. Examples of potential linker chemistries are hydrazide, ester, ether, and sulfhydryl. Amino caproic acid is an example of a bifunctional linker group. When amino caproic acid is used as part of the linker group, it is not counted as an amino acid in the numbering scheme of the oligopeptide.

The oligopeptide moiety is linked at a first attachment site of the oligopeptide to a stabilizing group that hinders cleavage of the oligopeptide by enzymes present in whole blood, and directly or indirectly linked to a therapeutic agent at a second attachment site of the oligopeptide. The linkage of the oligopeptide to the therapeutic agent and the stabilizing group may be performed in any order or concurrently. The resulting conjugate is tested for cleavability by TOP. Test compounds resistant to cleavage by TOP are selected. The resulting conjugate may also be tested for stability in whole blood. Test compounds stable in whole blood are selected.

The combination of oligopeptide, stabilizing group, and optional linker of U.S. Pat. No. 7,115,573 is further described in US 2002-0142955, also incorporated herein by reference.

In other embodiments of aspects of the invention, the compounds comprise those of Formula (Ib) and Formula (IIa) as defined above, except that R²⁰ of Z² (of Formula Ib) and Z³ (of Formula IIa) is described in US 2004-0019017 A1 (incorporated by reference in its entirety and which describes caspase inhibitor prodrugs), as comprising:

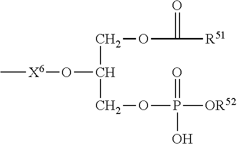

wherein R⁵¹ is a saturated or unsaturated, straight-chain or branched, substituted or unsubstituted alkyl of 2 to 30, preferably 2 to 24, carbon atoms;
R⁵² is H or a phospholipid head group, preferably choline;

X⁶ is a direct covalent bond or a group C(O)LR⁵³ wherein L is a saturated or unsaturated, straight-chain or branched, substituted or unsubstituted alkyl having from 2 to 15 carbon atoms, which optionally includes cyclic elements, and is optionally interrupted by one or more atoms selected from the group consisting of oxygen, sulfur and N(R⁵⁴); R⁵³ is selected from the group consisting of O, S and N(R⁵⁴), wherein R⁵⁴ is H or a saturated or unsaturated alkyl having 1 to 6 carbon atoms.

In certain embodiments of aspects of the invention, the compounds comprise those of Formula (Ib) and Formula (IIa) as defined above, except that R²⁰ of Z² (of Formula Ib) and Z³ (of Formula IIa) is the Y moiety described in U.S. Pat. No. 7,115,573 (incorporated by reference in its entirety).

In other embodiments of aspects of the invention, the compounds comprise those of Formula (Ib) and Formula (IIa) as defined above, except that R²⁰ of Z² (of Formula Ib) and Z³ (of Formula IIa) is described in US 2006-0166903 A1 (incorporated by reference in its entirety, as comprising-X-L-O—P(O)(O⁻)—O—CH₂—CH₂—N(CH₃)₃⁺, wherein X and L are as described in US 2006-0166903A1.

In other embodiments, the compounds of the invention comprise those of Formula (Ib) and Formula (IIa) as defined above, except Z² (of Formula Ib) and Z³ (of Formula IIa) is one of the cleavable prodrug moieties described in U.S. Pat. No. 6,855,702, US 2005-0137141, and US 2006-0135594, all hereby incorporated by reference in their entirety.

In certain embodiments of aspects of the present invention, the fungus or fungal unit thereof involved in infection and/or disease of keratinized tissue, such as onychomycosis, is a dermatophyte or a fungal unit thereof.

In certain embodiments of aspects of the present invention, the fungus is in an anamorph state. In certain embodiments, the fungus is in a teleomorph state.

In certain embodiments, the fungus is selected from Epidermophyton, Microsporum, and Trichophyton spp. and variants thereof. In certain embodiments, the fungus is from the genus Arthroderma (anamorph Microsporum, Trichophyton). In certain embodiments, the fungus is selected from the group consisting of Epidermophyton floccosum, Microsporum audouinii, M. canis, M. equinum, M. ferrugineum, M. fulvum, M. gallinae, M. gypseum, M. nanum, M. persicolor, M. praecox, M. racemosum, M. vanbreuseghemii, Trichophyton concentricum, T. equinum, T. gourvilii, T. kanei, T. megninii, T. mentagrophytes (including T. mentagrophytes var. interdigitale, T. mentagrophytes var. mentagrophytes), T. raubitschekii, T. rubrum, T. schoenleinii, T. simii, T. soudanense, T. tonsurans, T. verrucosum, T. violaceum and T. yaoundei. In certain embodiments, the fungus is selected from the group consisting of Arthroderma benhamiae (anamorph T. mentagrophytes), A. fulvum (anamorph M. fulvum), A. grubyi (anamorph M. vanbreuseghemii), A. gypseum (anamorph M. gypseum), A. incurvatum (anamorph M. gypseum), A. obtusum (anamorph M. nanum), A. otae (anamorph M. canis var. canis, M. canis var. distortum), A. persicolor (anamorph M. persicolor), A. simii (anamorph T. simii), A. racemosum (anamorph M. racemosum) and A. vanbreuseghemii (anamorph T. mentagrophytes).

In certain embodiments of aspects of the present invention, the fungus is selected from the group consisting of Epidermophyton floccosum, Trichophyton rubrum, T. tonsurans, T. mentagrophytes, T. verrucosum, T. schoenleinii, T. violaceum, T. equinum, Microsporum canis, M. audouinii, M. gypseum and M. nanum.

In certain embodiments of aspects of the present invention, the fungus is selected from the group consisting of Trichophyton tonsurans, T. schoenleinii, T. violaceum, Microsporum canis and M. audouinii.

In certain embodiments of aspects of the present invention, the fungus is selected from the group consisting of T. mentagrophytes, T. rubrum, and M. canis.

In certain embodiments of aspects of the present invention, the fungus is selected from the group consisting of T. rubrum, T. mentagrophytes and E. floccosum.

In certain embodiments of aspects of the present invention, the fungus is T. mentagrophytes or T. rubrum.

In certain embodiments of aspects of the present invention, the fungus is T. mentagrophytes.

In certain embodiments of aspects of the present invention, the fungus is T. rubrum.

In certain embodiments of aspects of the present invention, the fungus is selected from the group consisting of M. canis, M. gypseum, T. mentagrophytes, T. equinum, T. verrucosum, and M. nanum.

In certain embodiments of aspects of the present invention, the fungus is M. canis.

In certain embodiments of aspects of the present invention, the fungus is T. verrucosum.

In certain embodiments of aspects of the present invention, the fungus is T. equinum or M. equinum.

In certain embodiments of aspects of the present invention, the fungus is M. nanum.

In certain embodiments of aspects of the present invention, the fungus is M. gallinae.

In certain embodiments of aspects of the present invention, the fungus is T. tonsurans.

In certain embodiments of aspects of the present invention, the fungus is Epidermophyton floccosum.

In certain embodiments of aspects of the present invention, the infection and/or disease is caused by a non-dermatophyte.

In certain embodiments of aspects of the present invention the non-dermatophyte is selected from the group consisting of Acremonium spp., Aspergillus spp., Candida spp., Fusarium spp., Scopulariopsis brevicaulis, Onychocola canadensis and Scytalidium dimidiatum.

In certain embodiments of aspects of the present invention the non-dermatophyte is selected from the group consisting of Acremonium spp., Scopulariopsis brevicaulis, Onychocola canadensis and Scytalidium dimidiatum.

In certain embodiments of aspects of the present invention the non-dermatophyte is Scopulariopsis brevicaulis.

In certain embodiments of aspects of the present invention the non-dermatophyte is Onychocola canadensis.

In certain embodiments of aspects of the present invention, the infection and/or disease is selected from the group consisting of Tinea barbae, Tinea capitis, Tinea corporis, Tinea cruris, Tinea favosa, Tinea faciei, Tinea imbricata, Tinea manuum, Tinea nigra, Tinea pedis, Tinea unguium and onychomycosis.

In certain embodiments of aspects of the present invention, the infection and/or disease is Tinea corporis.

In certain embodiments of aspects of the present invention, the infection and/or disease is Tinea pedis.

In certain embodiments of aspects of the present invention, the infection and/or disease is onychomycosis.

In certain embodiments of aspects of the present invention, the infection and/or disease is candidal onychomycosis.

In certain embodiments of aspects of the present invention, the fungal histone deacetylase is represented by a nucleic acid comprising a gene selected from the group consisting of RPD3, HDA1, HOS1, HOS2, HOS3 and SIR2 and functional mutants, alleles and homologs thereof. In certain embodiments of aspects of the present invention, the fungal histone deacetylase is represented by the gene HOS2 or a functional mutant, allele or a homolog thereof. In certain embodiments of aspects of the present invention, the fungal histone deacetylase is represented by the gene HOS2.

In certain embodiments of aspects of the present invention, the fungal histone deacetylase is represented by a polypeptide selected from the group consisting of Rpd3, Hda1, Hos1, Hos2, Hos3 and Sir2 and functional mutants and homologs thereof. In certain embodiments of aspects of the present invention, the fungal histone deacetylase is represented by Hos2 or a functional mutant or homolog thereof. In certain embodiments of aspects of the present invention, the fungal histone deacetylase is represented by Hos2.

In certain embodiments of aspects of the invention, inhibition by an inhibitor of the activity of a histone deacetylase is specific; for example, the inhibitor of enzymatic activity of a histone deacetylase reduces the ability of a histone deacetylase to remove an acetyl group from a protein at a concentration that is lower than the concentration of the inhibitor that is required to produce another, unrelated biological effect. In certain embodiments, the concentration of the inhibitor required for inhibiting the activity of a histone deacetylase is at least 2-fold lower, in certain other embodiments at least 5-fold lower, in certain other embodiments at least 10-fold lower, and in certain other embodiments at least 20-fold lower than the concentration required to produce an unrelated biological effect.

In certain embodiments of aspects of the present invention the inhibitor of the activity of a histone deacetylase inhibits one or more fungal histone deacetylase, but less than all fungal histone deacetylase. In certain embodiments, the histone deacetylase inhibitor inhibits class I and class II histone deacetylase. In certain embodiments, the histone deacetylase inhibitor inhibits class I or class II histone deacetylase. In certain embodiments the inhibitor of the activity of a histone deacetylase inhibits one or more of Rpd3, Hos1, Hos2, Hda1, Hos3, Sir2 and Hst and functional mutants and homologs thereof. In certain embodiments, the inhibitor inhibits Hos2 and functional mutants and homologs thereof. In certain embodiments, the inhibitor is specific for Hos2 and homologs thereof; alternatively it is specific for Hos2.

In certain embodiments of aspects of the present invention, the antifungal agent is capable of preventing or treating a fungal infection in an animal, for example a human. In certain embodiments, the antifungal agent is a broad spectrum antifungal agent. In certain embodiments, the antifungal agent is specific to one or more particular species of fungus.

In certain embodiments of aspects of the present invention, the antifungal agent is an ergosterol synthesis inhibitor, such as but are not limited to an azole and phenpropimorph. In certain embodiments of aspects of the present invention, the antifungal agent is an azole. Other antifungal agents include, but are not limited to terbinafine. In certain embodiments, the azole is an imidazole or triazole. In certain embodiments, the antifungal agent is ketoconazole, itroconazole, fluconazole, voriconazole, posaconazole, ravuconazole or miconazole. In certain embodiments, the antifungal agent is fluconazole or itraconazole. In certain embodiments, the antifungal agent is fluconazole. Like azoles, phenpropimorph is an ergosterol synthesis inhibitor, but acts on the ergosterol reductase (ERG24) step of the synthesis pathway. Terbinafine, is also an ergosterol inhibitor, but acts on the squalene eposidase (ERG1) step.

Administration of a compound according to the present invention may be by any route appropriate, including, without limitation, parenteral, oral, sublingual, transdermal, topical, intranasal, intratracheal, intravenous or intrarectal. In certain embodiments of aspects of the present invention, administration is intravenously, for example in a hospital setting. In certain other embodiments of aspects of the present invention, compounds of the invention are administered topically or orally. In certain embodiments of aspects of the present invention, oral administration is, for example, via a capsule, liquid, drops, powder, tablet, lozenge, suspension or gel. In certain embodiments of aspects of the present invention, administration is topically. In certain embodiments of aspects of the present invention topical administration is, for example, via a cream, lacquer, ointment, powder, solution, paste, spray, shampoo, lotion or gel.

In certain embodiments of the present invention, the subject is an animal having growth thereon and/or therein of a fungus or fungal unit thereof involved in infection and/or disease of keratinized tissue, such as onychomycosis. In certain embodiments, the subject is an animal having infection and/or disease of keratinized tissue, such as onychomycosis. In certain embodiments, the subject is a mammal, for example a domesticated mammal such as but not limited to cattle, horse, sheep, goat, swine, dog or cat. In certain embodiments of the present invention, the subject is a human.

### DEFINITIONS

As used in the present specification, the following words and phrases are generally intended to have the meanings as set forth below, except to the extent that the context in which there are used indicates otherwise or they are expressly defined to mean something different.

The term “dermatophyte” is intended to refer to a group of closely related fungi that have the capacity to invade keratinized tissue of humans and/or other animals and produce an infection and/or disease.

In certain embodiments of aspects of the present invention, the term “fungus” or “fungal” is intended as a generic term to include a combination of more than one different fungal species, for example when referring to a group of different fungal species causing a fungal infection and/or disease.

Histone acetylation is a reversible modification, with deacetylation being catalyzed by a family of enzymes termed histone deacetylases (HDACs). The molecular cloning of gene sequences encoding proteins with HDAC activity has established the existence of a set of discrete HDAC enzyme isoforms. Yang and Grégoire, Mol. Cell. Biol. 25:2873-2884 (2005) teach that, based on phylogenetic analyses and sequence homology to yeast Rpd3 (reduced potassium dependency 3), Hda1 and Sir2 (silent information regulator 2), HDACs are grouped into distinct classes. In humans, for example, there are 18 known HDACs, which are divided into four classes: class I (HDAC1, -2, -3 and -8; homologous to Rpd3), class II (HDAC4, -5, -6, -7, -9 and -10; related to Hda1), class 111 (Sirt1, -2, -3, -4, -5, -6 and -7; similar to Sir2) and class IV (HDAC11). Class I, II and IV HDACs are zinc-dependent enzymes. Class III HDACs are NAD⁺ dependent deacetylases. In Saccharomyces cerevisiae, for example, there are 10 known HDACs, which are divided into three classes: class I (Rpd3, Hos1 and Hos2), class II (Hda1 and Hos3), and class III (Sir2 and four Hst proteins (Hst1 to Hst4), homologs of Sir2).

As used herein, the terms “histone deacetylase” and “HDAC” are intended to refer to any of a family of enzymes that remove acetyl groups from a protein (for example, a histone). Unless otherwise indicated by context, the term “histone” is meant to refer to any histone protein from any species. In certain embodiments of aspects of the present invention, the histone deacetylase is a mammalian, for example a human, histone deacetylase, including but not limited to HDAC1, HDAC2, HDAC3, HDAC4, HDAC5, HDAC6, HDAC7, HDAC8, HDAC9, HDAC10 and HDAC11. In certain embodiments, the histone deacetylase is a fungal histone deacetylase, including but not limited to RPD3, HDA1, HOS1, HOS2, HOS3 and SIR2. The terms RPD3, HDA1, HOS1, HOS2, HOS3 and SIR2, refer to those genes as they are known in the art, including those corresponding genes in fungi involved in infection and/or disease of keratinized tissue, such as onychomycosis, mutants and alleles thereof, and in certain embodiments where indicated are intended to further include any homologs thereof, respectively. The terms Rpd3, Hda1, Hos1, Hos2, Hos3 and Sir2, refer to encoded polypeptide products of such genes and in certain embodiments where indicated are intended to further include any mutants and homologs thereof, respectively.

The term “homolog” is a generic term used in the art and is intended to mean a polynucleotide or polypeptide sequence possessing a high degree of sequence relatedness to a reference sequence. Such relatedness may be quantified by determining the degree of identity and/or similarity between the two sequences as determined by those of skill in the art. Falling within this generic term are the terms “ortholog”, and “paralog”. “Ortholog” refers to a polynucleotide or polypeptide that is the functional equivalent of the polynucleotide or polypeptide in another species. “Paralog” refers to a polynucleotide or polypeptide within the same species which is functionally similar.

The present invention also encompasses allelic variants of histone deacetylase polypeptides and the nucleic acids encoding them; that is, naturally-occurring alternative forms of such polypeptides and nucleic acids in which differences in amino acid or nucleotide sequence are attributable to genetic polymorphism (allelic variation among individuals within a population). Naturally and artificially occurring HOS2 and Hos2 mutants are also encompassed by the present invention.

Homologs and alleles of a histone deacetylase can be identified by conventional techniques known to one skilled in the art. For example, a homolog of S. cerevisiae HOS2 may be isolated and identified by making suitable probes or primers from polynucleotides encoding HOS2 and screening a suitable nucleic acid source from the desired species, for example a cDNA library, and selecting positive clones. Thus, an aspect of the invention are nucleic acid sequences which encode for Hos2 homolog and allelic polypeptides and which hybridize under stringent conditions to a nucleic acid molecule comprising a sequence of nucleic acid corresponding to a region of nucleic acid encoding HOS2. The term “stringent conditions” as used herein refers to parameters with which the art is familiar.

The terms “histone deacetylase inhibitor”, “inhibitor of histone deacetylase” and “inhibitor of the activity of a histone deacetylase” and the like, are intended to mean either a compound which is capable of interacting with a polynucleotide encoding a product with histone deacetylase enzymatic activity and inhibiting the transcription and/or translation of the polynucleotide, or a compound which is capable of interacting with a polypeptide with histone deacetylase enzymatic activity and inhibiting histone deacetylase enzymatic activity. “Inhibiting histone deacetylase enzymatic activity” means reducing the ability of a histone deacetylase to remove an acetyl group from a protein, such as a histone. In certain embodiments, such reduction of histone deacetylase activity is at least about 50%, in certain embodiments, at least about 75%, and still certain other embodiments at least about 90%. In certain other embodiments, histone deacetylase activity is reduced by at least 95% and in certain other embodiments by at least 99%.

The term “hydroxamate-based” is intended to mean a compound includes a hydroxamate moiety.

The term “antifungal activity” is intended to mean the ability of a substance to inhibit or prevent, without limitation, the growth, viability and/or reproduction of a fungus or fungal unit thereof and/or the ability and/or degree to which a fungus or fungal unit thereof is capable of infecting a subject and/or causing disease in or on the subject.

The term “antifungal agent” is intended to mean a substance capable of inhibiting or preventing, without limitation, the growth, viability and/or reproduction of a fungus or fungal unit thereof, and/or the ability and/or degree to which a fungus or fungal unit thereof is capable of infecting a subject and/or causing disease in or on the subject.

A large number of active antifungal agents have an azole functionality as part of their structure; such an antifungal agent is generally referred to as an “antifungal azole”, an “azole antifungal agent” or an “azole”.

The term “keratin” is intended to refer to a family of fibrous structural proteins which form strong tissues found in, for example, reptiles, birds, amphibians, and mammals. The term “keratinized tissue” is intended to mean any tissue comprising or composed of keratin. Such tissues include those found in reptiles, birds, amphibians, and/or mammals, including by not limited to skin, hair, nails, claws, hooves, horns, wool, feathers, and teeth enamel.

The term “fungal unit” is intended to refer to any structural or reproductive unit of a fungus, which is capable of growth and/or causing infection and/or disease of a keratinized tissue. Examples of such fungal units include but, are not limited to, mycelium, hypae or viable fragment thereof, spore (including arthrospore and conidium, including microconidium and macroconidium, chlamydoconidium, chlamydospore, ascospore, arthroconidium) conidiophore, ascocarp, ascus.

The term “effective amount” is intended to mean an amount of a substance that achieves the effect which is intended with its application. The amount of a compound of the invention, for example, which constitutes an “effect amount” will vary depending on the compound, the intended use, the fungus, whether the fungus is in vitro or in vivo, if in vivo then the species of the subject in/on which its use is desired, and the like. The effective amount can be determined routinely by one of ordinary skill in the art.

The term “subject” is intended to mean humans and other animals, such as birds, reptiles, amphibians and other mammals, including domesticated animals. Thus, the compounds, compositions and methods of the present invention are applicable to both human and veterinary applications.

The term “sensitizing a fungus or fungal unit thereof to an antifungal agent” is intended to mean increasing the sensitivity of a fungus or fungal unit thereof to the antifungal agent. Sensitivity can be determined, for example, by measuring killing of the fungus or fungal unit thereof, inhibition of growth of the fungus or fungal unit thereof, increase of surrogate markers for death, or decrease in surrogate markers for growth of the fungus or fungal unit thereof.

The present invention also provides kits, which may be used in the methods described herein. As used herein, the term “kit” refers to a component or set of components, for the purpose of performing a method, such as those described herein. Kits can include a means for delivery of, for example, a compound according to the present invention, or another antifungal agent(s) or mixtures thereof such as a syringe for injection, pressure pack for capsules, apparatus for intravenous administration, spray bottle or applicator for topical administration, and the like. A kit can provide a reagent(s) to prepare a composition comprising, for example, a compound according to the present invention for administration. The compound can be in a dry or lyophilized form or in a solution, particularly a sterile solution. When the compound is in a dry form, the kit optionally includes a pharmaceutically acceptable diluent for preparing a liquid formulation. The kit can include another therapeutic compound for use in combination with the compounds described herein. Such other therapeutic compound can be provided in a separate form or mixed with a compound described herein. In certain embodiments of aspects of the present invention, the kit provides the necessary ingredients with instructions such that one of ordinary skill in the art can combine the ingredients into an appropriate dosage form for delivery to a subject.

A kit optionally includes appropriate instructions for preparation and administration of the active ingredient(s), and any other relevant information. The instructions can be in any suitable format, including, but not limited to, printed matter, videotape, computer readable disk or optical disc.

The compounds, synergistic combinations thereof, methods and kits of the present invention can be used in a variety of applications, including, for example, medicines and treatments for treating fungal infections and/or diseases of keratinized tissue, such as onychomycosis of animals, including humans; or as part of a cleaning and/or sterilization regimen, such as in a disinfectant formulation, (for example wherein a cleaning solution comprises a compound and/or synergistic combination thereof), for laboratory, medical or veterinarial tools and equipment, animal housings, medical or veterinarial laundry, shoe disinfection, hospitals, operating and examination rooms, hospital beds, etc. As such, the present invention is in no way intended to be limited to purely human applications and is intended to encompass for example, veterinary application, including methods for treating fungal infection and/or diseases of keratinized tissue, such as onychomycosis, of non-human animals.

The terms “selective”, “selectively” and “selectivity”, as used throughout herein, are intended to mean that the compounds and/or inhibitors as described herein and their use in the compositions and methods described herein achieve their purpose without being used in concentrations that are toxic to host cells. “Host cells” are the cells of subject to be treated.

A minimum inhibitory concentration (MICₓ) of a compound such as a HDAC inhibitor or an antifungal agent (or both) is the concentration that reduces growth of a fungus by X %, compared to the growth of the fungus in the absence of the compound.

Mammalian cytotoxicity of a compound such as an HDAC inhibitor can be determined, for example, by the reduction of 3-(4,5-dimethylthiazol-2-yl)-2,5-diphenyltetrazolium bromide (MTT; Sigma), measured as absorption at 570 nm.

The terms “treating”, “treatment”, or the like, as used herein covers the treatment of a disease-state in an animal, and includes at least one of: (i) preventing the disease-state from occurring in an animal, in particular, when such animal is predisposed to the disease-state but has not yet been diagnosed as having it; (ii) inhibiting the disease-state, i.e., partially or completely arresting its development; (iii) relieving the disease-state, i.e., causing regression of symptoms of the disease-state or ameliorating a symptom of the disease; and (iv) reversal or regression of the disease-state, such as eliminating or curing of the disease. In a certain embodiments of the present invention the animal is a mammal, such as a human. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). On occasion a moiety may be defined, for example, as (A)ₐ-B—, wherein a is 0 or 1. In such instances, when a is 0 the moiety is B— and when a is 1 the moiety is A-B—.

For simplicity, reference to a “Cₙ-Cₘ” heterocyclyl or “Cₙ-Cₘ” heteroaryl means a heterocyclyl or heteroaryl having from “n” to “m” annular atoms, where “n” and “m” are integers. Thus, for example, a C₅-C₆-heterocyclyl is a 5- or 6-membered ring having at least one heteroatom, and includes pyrrolidinyl (C₅) and piperidinyl (C₆); C₆-heteroaryl includes, for example, pyridyl and pyrimidyl.

The term “alkyl” is intended to mean a straight or branched chain aliphatic group having from 1 to 12 carbon atoms. In certain embodiments of aspects of the present invention, the alkyl has 1-8 carbon atoms, and in certain embodiments 1-6 carbon atoms. In certain embodiments of aspects of the present invention alkyl groups have from 2 to 12 carbon atoms; in certain embodiments 2-8 carbon atoms; and in certain embodiments 2-6 carbon atoms. Examples of alkyl groups include, without limitation, methyl, ethyl, propyl, isopropyl, butyl, isobutyl, sec-butyl, tert-butyl, pentyl, and hexyl. A “C₀” alkyl (as in “C₀-C₃-alkyl”) is a covalent bond.

The term “alkenyl” is intended to mean an unsaturated straight or branched chain aliphatic group with one or more carbon-carbon double bonds, having from 2 to 12 carbon atoms. In certain embodiments of aspects of the present invention, the alkenyl has 2-8 carbon atoms; and in certain embodiments 2-6 carbon atoms. Examples of alkenyl groups include, without limitation, ethenyl, propenyl, butenyl, pentenyl, and hexenyl.

The term “alkynyl” is intended to mean an unsaturated straight or branched chain aliphatic group with one or more carbon-carbon triple bonds, having from 2 to 12 carbon atoms. In certain embodiments of aspects of the present invention, the alkynyl has 2-8 carbon atoms, and in certain embodiments 2-6 carbon atoms. Examples of alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. In certain embodiments of aspects of the present invention alkylene groups include, without limitation, methylene, ethylene, propylene, and butylene. In certain embodiments of aspects of the present invention alkenylene groups include, without limitation, ethenylene, propenylene, and butenylene. In certain embodiments of aspects of the present invention alkynylene groups include, without limitation, ethynylene, propynylene, and butynylene.

The term “cycloalkyl” is intended to mean a saturated or unsaturated mono-, bi, tri- or poly-cyclic hydrocarbon group having about 3 to 15 carbons, alternatively having 3 to 12 carbons, alternatively 3 to 8 carbons, and alternatively 3 to 6 carbons. In certain embodiments, the cycloalkyl group is fused to an aryl, heteroaryl or heterocyclic group. Examples of cycloalkyl groups include, without limitation, cyclopenten-2-enone, cyclopenten-2-enol, cyclohex-2-enone, cyclohex-2-enol, cyclopropyl, cyclobutyl, cyclopentyl, cyclopentenyl, cyclohexyl, cyclohexenyl, cycloheptyl, and cyclooctyl.

The terms “heterocyclyl”, “heterocyclic” or “heterocycle” are intended to mean a group which is a mono-, bi-, or polycyclic structure having from about 3 to about 14 atoms, wherein one or more atoms are independently selected from the group consisting of N, O, and S. The ring structure may be saturated, unsaturated or partially unsaturated. In certain embodiments, the heterocyclic group is non-aromatic. In a bicyclic or polycyclic structure, one or more rings may be aromatic; for example one ring of a bicyclic heterocycle or one or two rings of a tricyclic heterocycle may be aromatic, as in indan and 9,10-dihydro anthracene. Examples of heterocyclic groups include, without limitation, epoxy, aziridinyl, tetrahydrofuranyl, pyrrolidinyl, piperidinyl, piperazinyl, thiazolidinyl, oxazolidinyl, oxazolidinonyl, and morpholino. In certain embodiments, the heterocyclic group is fused to an aryl, heteroaryl, or cycloalkyl group. Examples of such fused heterocycles include, without limitation, tetrahydroquinoline and dihydrobenzofuran. Specifically excluded from the scope of this term are compounds where an annular O or S atom is adjacent to another O or S atom.

In certain embodiments, the heterocyclic group is a heteroaryl group. As used herein, the term “heteroaryl” is intended to mean a mono-, bi-, tri- or polycyclic group having 5 to 14 ring atoms, preferably 5, 6, 9, or 10 ring atoms; having 6, 10, or 14 pi electrons shared in a cyclic array; and having, in addition to carbon atoms, between one or more heteroatoms independently selected from the group consisting of N, O, and S. For example, a heteroaryl group may be pyrimidinyl, pyridinyl, benzimidazolyl, thienyl, benzothiazolyl, benzofuranyl and indolinyl. In certain embodiments of aspects of the present invention heteroaryl groups include, without limitation, thienyl, benzothienyl, furyl, benzofuryl, dibenzofuryl, pyrrolyl, imidazolyl, pyrazolyl, pyridyl, pyrazinyl, pyrimidinyl, indolyl, quinolyl, isoquinolyl, quinoxalinyl, tetrazolyl, oxazolyl, thiazolyl, and isoxazolyl.

The term “aryl” is intended to mean a mono-, bi-, tri- or polycyclic C₆-C₁₄ aromatic moiety, comprising one to three aromatic rings. In certain embodiments of aspects of the present invention, the aryl group is a C₆-C₁₀ aryl group, for example a C₆ aryl group. Examples of aryl groups include, without limitation, phenyl, naphthyl, anthracenyl, and fluorenyl.

In certain embodiments of aspects of the present invention heterocyclyls and heteroaryls include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, thiadiazolyl (e.g., 1,2,3-thiadiazolyl, 1,2,4-thiadiazolyl, 1,2,5-thiadiazolyl, 1,3,4-thiadiazolyl), thianthrenyl, thiazolyl, thienyl, thienothiazolyl, thienooxazolyl, thienoimidazolyl, thiophenyl, triazinyl, triazolyl (e.g., 1,2,3-triazolyl, 1,2,4-triazolyl, 1,2,5-triazolyl. 1,3,4-triazolyl), and xanthenyl.

As employed herein, and unless stated otherwise, when a moiety (e.g., alkyl, heteroalkyl, cycloalkyl, aryl, heteroaryl, heterocyclyl, etc.) is described as “optionally substituted” it is meant that the group optionally has from one to four, alternatively from one to three, alternatively one or two, non-hydrogen substituents. Suitable substituents include, without limitation, halo, hydroxy, oxo (e.g., an annular —CH— substituted with oxo is —C(O)—) nitro, halohydrocarbyl, hydrocarbyl, alkyl, cycloalkyl, heterocyclyl, aryl, heteroaryl, aralkyl, alkoxy, aryloxy, amino, acylamino, alkylcarbamoyl, arylcarbamoyl, aminoalkyl, acyl, carboxy, hydroxyalkyl, alkanesulfonyl, arenesulfonyl, alkanesulfonamido, arenesulfonamido, aralkylsulfonami do, alkylcarbonyl, acyloxy, cyano, and ureido groups. In certain embodiments of aspects of the present invention, substituents, which are themselves not further substituted (unless expressly stated otherwise) are:
(a) halo, cyano, oxo, carboxy, formyl, nitro, amino, amidino, guanidino,
(b) C₁-C₅ alkyl or alkenyl or arylalkyl imino, carbamoyl, azido, carboxamido, mercapto, hydroxy, hydroxyalkyl, alkylaryl, arylalkyl, C₁-C₈ alkyl, C₁-C₈ alkenyl, C₁-C₈ alkoxy, C₁-C₈ alkoxycarbonyl, aryloxycarbonyl. C₂-C₈ acyl, C₂-C₈ acylamino, C₁-C₈ alkylthio, arylalkylthio, arylthio, C₁-C₈ alkylsulfinyl, arylalkylsulfinyl, arylsulfinyl, C₁-C₈ alkylsulfonyl, arylalkylsulfonyl, arylsulfonyl, C₀-C₆ N-alkyl carbamoyl, C₂-C₁₅ N,N-dialkylcarbamoyl, C₃-C₇ cycloalkyl, aroyl, aryloxy, arylalkyl ether, aryl, aryl fused to a cycloalkyl or heterocycle or another aryl ring, C₃-C₇ heterocycle, C₅-C₁₅ heteroaryl or any of these rings fused or spiro-fused to a cycloalkyl, heterocyclyl, or aryl, wherein each of the foregoing is further optionally substituted with one more moieties listed in (a), above; and
(c) —(CR³²R³³)ₛ—NR³⁰R³¹, wherein s is from 0 (in which case the nitrogen is directly bonded to the moiety that is substituted) to 6, R³² and R³³ are each independently hydrogen, halo, hydroxyl or C₁-C₄alkyl, and R³⁰ and R³¹ are each independently hydrogen, cyano, oxo, hydroxyl, —C₁-C₈ alkyl, C₁-C₅ heteroalkyl, C₁-C₈ alkenyl, carboxamido, C₁-C₃ alkyl-carboxamido, carboxamido-C₁-C₃ alkyl, amidino, C₂-C₈hydroxyalkyl, C₁-C₃ alkylaryl, aryl-C₁-C₃ alkyl, C₁-C₃ alkylheteroaryl, heteroaryl-C₁-C₃ alkyl. C₁-C₃ alkylheterocyclyl, heterocyclyl-C₁-C₃ alkyl C₁-C₃ alkylcycloalkyl, cycloalkyl-C₁-C₃ alkyl, C₂-C₈ alkoxy, C₂-C₈ alkoxy-C₁-C₄alkyl, C₁-C₅ alkoxycarbonyl, aryloxycarbonyl, aryl-C₁-C₃ alkoxycarbonyl, heteroaryloxycarbonyl, heteroaryl-C₁-C₃ alkoxycarbonyl, C₁-C₈ acyl, C₀-C₈ alkyl-carbonyl, aryl-C₀-C₈ alkyl-carbonyl, heteroaryl-C₀-C₈ alkyl-carbonyl, cycloalkyl-C₀-C₈ alkyl-carbonyl, C₀-C₈ alkyl-NH-carbonyl, aryl-C₀-C₈ alkyl-NH-carbonyl, heteroaryl-C₀-C₈ alkyl-NH-carbonyl, cycloalkyl-C₀-C₈ alkyl-NH-carbonyl, C₀-C₈ alkyl-O-carbonyl, aryl-C₀-C₈ alkyl-O-carbonyl, heteroaryl-C₀-C₈ alkyl-O-carbonyl, cycloalkyl-C₀-C₈ alkyl-O-carbonyl, C₁-C₈ alkylsulfonyl, arylalkylsulfonyl, arylsulfonyl, heteroarylalkylsulfonyl, heteroarylsulthnyl, C₁-C₈ alkyl-NH-sulfonyl, arylalkyl-NH-sulfonyl, aryl-NH-sulfonyl, heteroarylalkyl-NH-sulfonyl, heteroaryl-NH-sulfonyl aroyl, aryl, cycloalkyl, heterocyclyl, heteroaryl, aryl-C₁-C₃ alkyl-, cycloalkyl-C₁-C₃ alkyl-, heterocyclyl-C₁-C₃ alkyl-, heteroaryl-C₁-C₃ alkyl-, or protecting group, wherein each of the foregoing is further optionally substituted with one more moieties listed in (a), above; or
R³⁰ and R³¹ taken together with the N to which they are attached form a heterocyclyl or heteroaryl, each of which is optionally substituted with from 1 to 3 substituents selected from the group consisting of (a) above, a protecting group, and (X³⁰—Y³¹—), wherein said heterocyclyl may also be bridged (forming a bicyclic moiety with a methylene, ethylene or propylene bridge); wherein
X³⁰ is selected from the group consisting of C₁-C₈alkyl, C₂-C₈alkenyl-, C₂-C₈alkynyl-, -C₀-C₃alkyl -C₂-C₈alkenyl-C₀-C₃alkyl, C₀-C₃alkyl-C₂-C₈alkynyl-C₀-C₃alkyl, C₀-C₃alkyl-O—C₀-C₃alkyl-, HO—C₀-C₃alkyl-, C₀-C₄alkyl-N(R³⁰)—C₀-C₃alkyl-, N(R³⁰)(R³¹)—C₀-C₃alkyl-, N(R³⁰)(R³¹)—C₀-C₃alkenyl-, N(R³⁰ (R³¹)—C₀-C₃alkynyl-, (N(R³⁰)(R³¹))₂—C═N—, C₀-C₃alkyl-S(O)₀₋₂—C₀-C₃alkyl-, CF₃—C₀-C₃alkyl-, C₁-C₈heteroalkyl, aryl, cycloalkyl, heterocyclyl, heteroaryl, aryl-C₁-C₃alkyl-, cycloalkyl-C₁-C₃alkyl-, heterocyclyl-C₁-C₃alkyl-, heteroaryl-C₁-C₃alkyl-, N(R³⁰)(R³¹)-heterocyclyl-C₁-C₃alkyl-, wherein the aryl, cycloalkyl, heteroaryl and heterocyclyl are optionally substituted with from 1 to 3 substituents from (a); and Y³¹ is selected from the group consisting of a direct bond, —O—, —N(R³⁰)—, —C(O)—, —O—C(O)—, —C(O)—O—, —N(R³⁰)—C(O)—, —C(O)—N(R³⁰)—, —N(R³⁰)—C(S)—, —C(S)—N(R³⁰)—, N(R³⁰)—C(O)—N(R³¹)—, —N(R³⁰)—C(NR³⁰)—N(R³¹)—, —N(R³⁰)—C(NR³¹)—, —C(NR³¹)—N(R³⁰), —N(R³⁰)—C(S)—N(R³¹)—, —N(R³⁰)—C(O)—O—, —O—C(O)—N(R³¹)—, —N(R³⁰)—C(S)—O—, —O—C(S)—N(R³¹)—, —S(O)₀₋₂—, —SO₂N(R³¹)—, —N(R³¹)—SO₂— and —N(R³⁰)—SO₂N(R³¹)—.

When there are two optional substituents bonded to adjacent atoms of a ring structure, such as for example phenyl, thiophenyl, or pyridinyl, the substituents, together with the atoms to which they are bonded, optionally form a 5- or 6-membered cycloalkyl or heterocycle having 1, 2, or 3 annular heteroatoms.

In certain embodiments, a heterocyclic group is substituted on carbon, nitrogen and/or sulfur at one or more positions. Examples of substituents on nitrogen include, but are not limited to N-oxide, alkyl, aryl, aralkyl, alkylcarbonyl, alkylsulfonyl, arylcarbonyl, arylsulfonyl, alkoxycarbonyl, or aralkoxycarbonyl. Examples of substituents on sulfur include, but are not limited to, oxo and C₁₋₆alkyl.

In addition, substituents on cyclic moieties (i.e., cycloalkyl, heterocyclyl, aryl, heteroaryl) include 5-6 membered mono- and 9-14 membered bi-cyclic moieties fused to the parent cyclic moiety to form a bi- or tri-cyclic fused ring system. Substituents on cyclic moieties also include 5-6 membered mono- and 9-14 membered bi-cyclic moieties attached to the parent cyclic moiety by a covalent bond to form a bi- or tri-cyclic bi-ring system. For example, an optionally substituted phenyl includes, but is not limited to, the following:

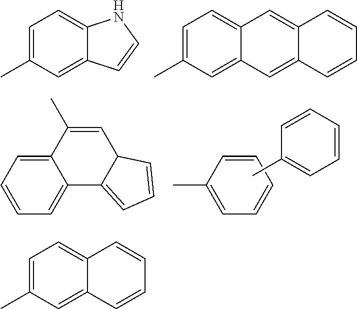

The term “pharmaceutically acceptable carrier” is intended to mean a non-toxic material that is compatible with a biological system in a cell, cell culture, tissue sample or body and that does not interfere with the effectiveness of the biological activity of the active ingredient(s). Thus, compositions according to the invention may contain, in addition to the inhibitor and antifungal agent, diluents, excipients, fillers, salts, buffers, stabilizers, solubilizers, and/or other materials well known in the art. Examples of the preparation of pharmaceutically acceptable formulations are described in, e.g., Remington's Pharmaceutical Sciences, 18th Edition, ed. A. Gennaro, Mack Publishing Co., Easton, Pa., 1990.

It will be understood that the characteristics of the carrier, will depend on the route of administration for a particular application.

The term “pharmaceutically acceptable salt” is intended to mean a salt that retains the desired biological activity of a compound of the present invention and exhibits minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be in the form of pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). As used herein, the term “salt” is also meant to encompass complexes, such as with an alkaline metal or an alkaline earth metal.

The active compounds of a composition of the invention are included in the pharmaceutically acceptable carrier in an amount sufficient to deliver an effective desired amount without causing serious toxic effects to an individual to which the composition is administered.

The present invention also includes prodrugs of compounds of the invention. The term “prodrug” is intended to represent covalently bonded carriers, which are capable of releasing the active ingredient when the prodrug is administered to a mammalian subject, or to a fungal cell. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of the invention include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups), amides (e.g., trifluoroacetylamino, acetylamino, and the like), and the like.

The compounds of the invention may be administered, for example, as is or as a prodrug, for example in the form of an in vivo hydrolyzable ester or in vivo hydrolyzable amide. An in vivo hydrolyzable ester of a compound of the invention containing a carboxy or hydroxy group is, for example, a pharmaceutically acceptable ester which is hydrolyzed in the organism being treated, preferably a human or animal body, to produce the parent acid or alcohol. Alternatively, hydrolization occurs in a fungal cell. Suitable pharmaceutically acceptable esters for carboxy include C₁₋₆-alkoxymethyl esters (e.g., methoxymethyl), C₁₋₆-alkanoyloxymethyl esters (e.g., for example pivaloyloxymethyl), phthalidyl esters, C₃₋₈-cycloalkoxycarbonyloxyC₁₋₆-alkyl esters (e.g., 1-cyclohexylcarbonyloxyethyl); 1,3-dioxolen-2-onylmethyl esters (e.g., 5-methyl-1,3-dioxolen-2-onylmethyl; and C₁₋₆-alkoxycarbonyloxyethyl esters (e.g., 1-methoxycarbonyloxyethyl) and may be formed at any appropriate carboxy group in the compounds of this invention.

An in vivo hydrolyzable ester of a compound of the invention containing a hydroxy group includes inorganic esters such as phosphate esters and α-acyloxyalkyl ethers and related compounds which as a result of the in vivo hydrolysis of the ester breakdown to give the parent hydroxy group. Examples of α-acyloxyalkyl ethers include acetoxymethoxy and 2,2-dimethylpropionyloxy-methoxy. A selection of in vivo hydrolyzable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(N,N-dialkylaminoethyl)-N-alkylcarbamoyl (to give carbamates), N,N-dialkylaminoacetyl and carboxyacetyl. Examples of substituents on benzoyl include morpholino and piperazino linked from a ring nitrogen atom via a methylene group to the 3- or 4-position of the benzoyl ring. A suitable value for an in vivo hydrolyzable amide of a compound of the invention containing a carboxy group is, for example, a N—C₁₋₆-alkyl or N,N-di-C₁₋₆-alkyl amide such as N-methyl, N-ethyl. IV-propyl, N,N-dimethyl, N-ethyl-N-methyl or N,N-diethyl amide.

The present invention is also directed to solvates and hydrates of the compounds of the present invention. The term “solvate” refers to a molecular complex of a compound with one or more solvent molecules in a stoichiometric or non-stoichiometric amount. A molecular complex of a compound or moiety of a compound and a solvent can be stabilized by non-covalent intra-molecular forces such as, for example, electrostatic forces, van der Waals forces, or hydrogen bonds. Those skilled in the art of organic chemistry will appreciate that many organic compounds can form such complexes with solvents in which they are obtained, prepared or synthesized, or from which they are precipitated or crystallized. The term “hydrate” refers to a complex in which the one or more solvent molecules are water and includes monohydrates, hemi-hydrates, dihydrates, hexahydrates, and the like. The meaning of the words “solvate” and “hydrate” are well known to those skilled in the art. Techniques for the preparation of solvates are well established in the art (see, for example, Brittain, Polymorphism in Pharmaceutical solids. Marcel Dekker, New York, 1999; Hilfiker, Polymorphism in the Pharmaceutical Industry, Wiley, Weinheim, Germany, 2006).

In some embodiments of this aspect, the solvent is an inorganic solvent (for example, water). In some embodiments of this aspect, the solvent is an organic solvent (such as, but not limited to, alcohols, such as, without limitation, methanol, ethanol, isopropanol, and the like, acetic acid, ketones, esters, and the like). In certain embodiments, the solvent is one commonly used in the pharmaceutical art, is known to be innocuous to a recipient to which such solvate is administered (for example, water, ethanol, and the like) and does not interfere with the biological activity of the solute.

The present invention will be more readily understood by referring to the following examples, which are given to illustrate the invention rather than to limit its scope.

### Example 1

This example describes the in vitro activity of combinations of compounds according to the present invention and antifungal agents against a collection of dermatophytic fungal pathogens. Antifungal susceptibility testing was adapted from the CLSI M38-A broth microdilution method for dermatophytes (NCCLS-M38-A2 2008). Combination testing was performed by checkerboard method (with compound 8 in combination with fluconazole, itraconazole or terbinafine). A total of 30 isolates of 5 different fungal species were tested in the combination test: 6 isolates each of Trichophyton tonsurans, Trichophyton rubrum, Epidermophyton floccosum, Trichophyton mentagrophytes and Microsporum canis. Clinical isolates were obtained by the Laboratory of Dr. M. Ghannoum at Case Western University.

Synergy was assessed using the fractional inhibitory concentration (FIC), an interaction coefficient, with the following definitions:

FIC<0.5=Synergy;

0.5≦FIC≦1.0=Additivity;

1.0<FIC≦4.0=No Interaction; and

FIC>4.0=Antagonistic.

The fold reduction of the itraconazole minimum inhibitory concentration (MIC) was also evaluated.

### Results

### Single Agent Activity of Compound 8 in Clinical Isolates of Dermatophytes

Results for the single agent activity of Compound 8 against clinical isolates of dermatophytes are shown in Table 1.

TABLE 1
Single Agent Activity of Compound 8 in Dermatophytes

| Organism | N | Cpd 8 MIC (μg/mL) |
|---|---|---|
| T. rubrum | 10 | 0.06-00.25 |
| T. mentagrophytes | 10 | 0.12-2 |
| E. floccosum | 9 | 0.5-1 |
| T. tonsurans | 8 | 0.02-0.25 |
| M. canis | 7 | 0.12-2 |

Compound 8 had single agent antifungal activity against the broad panel of dermatophytic clinical isolates (MIC values ranged from 0.02 to 2 μg/mL).

Activity of Compound 8 in Combination with Antifungals in Clinical Isolates of Dermatophytes
Results of synergistic activity of Compound 8 in combination with fluconazole, itraconazole or terbinafine when examined against the same broad panel of clinical isolates of dermatophytes are shown in Table 2

TABLE 2
Activity of Compound 8 in Combination against Clinical Isolates of Dermatophytes

| | Synergy | | | Additivity | | | No Interaction | | |
|---|---|---|---|---|---|---|---|---|---|
| Organism | # isolates (%) | | | # isolates (%) | | | # isolates (%) | | |
| (n = 6 per azole | Itracon- | Flucon- | Terbin- | Itracon- | Flucon- | Terbin- | Itracon- | Flucon- | Terbin- |
| group) | azole | azole | afine | azole | azole | afine | azole | azole | afine |
| T. rubrum | 4 | 2 | † | 2 | 4 | † | 0 | 0 | † |
| | (67%) | (33%) | | (33%) | (67%) | | | | |
| T. mentagrophytes | 5 | 2 | † | 1 | 4 | † | 0 | 0 | † |
| | (83%) | (33%) | | (17%) | (67%) | | | | |
| E. floccosum | 4 | 0 | † | 2 | 4 | † | 0 | 2 | † |
| | (67%) | | | (33%) | (67%) | | | (33%) | |
| T. tonsurans | 1 | 5 | † | 4 | 1 | † | 0 | 0 | † |
| | (20%)* | (83%) | | (80%)* | (17%) | | | | |
| M. canis | 1 | 1 | † | 0* | 4 | † | 4 | 1 | † |
| | (20%)* | (17%) | | | (67%) | | (80%)* | (17%) | |

*N = 5 for itraconazole group
† = synergy could not be determined because of the high potency (extremely low MIC) of terbinafine
Synergy was observed between Compound 8 and itraconazole (in 15 of 28, or 54% of the isolate combinations tested with itraconazole), as well as between Compound 8 and fluconazole (in 10 of 30, or 33% of the isolate combinations tested with fluconazole) against the panel of clinical isolates of 5 common dermatophytic fungi. Synergy could not be determined between Compound 8 and terbinafine in these organisms because of the high potency (i.e. the extremely low MIC) of terbinafine.

Results for the reduction of the MIC of itraconazole in clinical isolates of dermatophytes are shown in Table 3

TABLE 3
Combination of Compound 8 with Itraconazole Lowers the MIC of
Itraconazole in Clinical Isolates of Dermatophytes

| | | | | Itraconazole | Fold Reduction of |
|---|---|---|---|---|---|
| | | Itraconazole | Cpd 8 MIC | MIC with Cpd 8 | Itraconazole MIC |
| Organism | Strain # | MIC (μg/mL) | (μg/mL) | (μg/mL) | by Cpd 8 |
| T. rubrum | 1 | 0.06 | 0.25 | 0.002 | 30x |
| | 2 | 0.25 | 0.03 | 0.004 | 63x |
| | 3 | 0.12 | 0.03 | 0.004 | 30x |
| | 4 | 0.25 | 0.25 | 0.06 | 4x |
| | 5 | 0.25 | 0.06 | 0.03 | 8x |
| | 6 | 0.25 | 0.25 | 0.002 | 125x |
| T. | 1 | 0.06 | 0.5 | 0.008 | 7.5x |
| mentagrophytes | 2 | 0.03 | 0.12 | 0.008 | 4x |
| | 3 | 0.12 | 1 | 0.03 | 4x |
| | 4 | 0.06 | 1 | 0 | — |
| | 5 | 0.25 | 1 | 0.015 | 16x |
| | 6 | 0.12 | 0.5 | 0.015 | 8x |
| E. floccosum | 1 | 0.12 | 0.12 | 0.015 | 8x |
| | 2 | 0.03 | 0.12 | 0.008 | 4x |
| | 3 | 0.06 | 0.5 | 0.008 | 7.5x |
| | 4 | 0.06 | 0.25 | 0.008 | 7.5x |
| | 5 | 0.06 | 0.25 | 0.015 | 4x |
| | 6 | 0.06 | 0.5 | 0.015 | 4x |
| T. tonsurans | 1 | 0.002 | 0.002 | 0.001 | 2x |
| | 2 | 0.002 | 0.03 | 0.001 | 2x |
| | 3 | 0.5 | 0.03 | 0.12 | 4x |
| | 4 | — | — | — | — |
| | 5 | 0.002 | 0.03 | 0.001 | 2x |
| | 6 | 0.002 | 0.03 | 0.001 | 2x |
| M. canis | 1 | 0.12 | 0.25 | 0.03 | 4x |
| | 2 | 0.008 | 1 | 0.008 | 0 |
| | 3 | 0.12 | 0.25 | 0.25 | 0 |
| | 4 | 0.06 | 0.5 | 0.03 | 2x |
| | 5 | 0.03 | 0.5 | 0.008 | 4x |

The combination of Compound 8 with itraconazole lowered the MIC of itraconazole by 4 to 125- fold against T. rubrum, by 4 to 16-fold against T. mentagrophytes, by 4 to 8-fold against E. floccosum, and by 0 to 4-fold against M. canis.

Compound 8 demonstrated single agent activity against all species tested with MIC values of 0.02-2 μg/mL. Compound 8 showed synergy with itraconazole against 15 of 28 isolates tested, and with fluconazole against 10 of 30 clinical isolates tested. Synergy could not be determined between Compound 8 and terbinafine in these organisms because of the high potency (extremely low MIC) of terbinafine. Antagonism was not observed for any of the Compound 8-azole combinations or clinical isolates. Compound 8 demonstrated in vitro synergy with azoles against the majority of clinical isolates of Zygomycetes and molds.

### Example 2

This example describes the in vitro activity of combinations of compounds according to the present invention and antifungal agents against clinical isolates of Trichophyton rubrum. Twenty clinical isolates Trichophyton rubrum, including 9 strains with elevated terbinafine MICs, taken from the culture collection of the Center for Medical Mycology, were tested.

### Methods

MIC testing was performed according to the modification of the CLSI M38-A2 standard for dermatophytes developed at the Center (CLSI. Reference Method for Broth Dilution Antifungal Susceptibility Testing of Filamentous Fungi; Approved Standard-Second Edition. CLSI document M38-A2 [ISBN 1-56238-668-9]. CLSI, 940 West Valley Road, Suite 1400, Wayne, Pa. 19087-1898 USA, 2008; Ghannoum M A, et al. Intra- and Interlaboratory Study of a Method for Testing the Antifungal Susceptibilities of Dermatophytes. 2004 J Clin Microbiol 42:(7): 2977-2979). Compound 8 and fluconazole were tested in combination using a checkerboard microdilution method. The interaction between Compound 8 and fluconazole was determined by FIC Index, or Fractional Inhibitory Concentration Index, which assigns a numerical value to the interaction of the two antifungals. Interpretation of the FIC Index was as follows:

| | | |
|---|---|---|
| MIC drug A in combination | + | MIC drug B in combination |
| MIC drug A alone | | MIC drug B alone |

FIC < 0.5 = Synergy;
0.5 ≦ FIC ≦ 1.0 = Additivity;
1.0 < FIC ≦ 4.0 = No Interaction; and
FIC > 4.0 = Antagonistic.

### Results and Discussion

Table 4 shows that the range of MIC values for Compound 8 against all of the T. rubrum strains tested. The MIC₅₀ (defined as the lowest concentration to inhibit 50% of the strains tested) of Compound 8 was equivalent to that of fluconazole, with values of both antifungals equal to 1.0 μg/ml. The Compound 8 MIC was lower than the corresponding terbinafine MIC for eight of nine strains with a terbinafine MIC≧4.0 μg/ml (Table 5a).

The MIC values for Compound 8 against T. mentagrophytes were lower than fluconazole with MIC₅₀ values of 1.0 μg/ml and 16 μg/ml respectively; similarly Compound 8 against T. tonsurans demonstrated lower MIC values than fluconazole, with an MIC₅₀ values of 0.06 μg/ml and 2.0 μg/ml respectively. For all T. mentagrophytes and T. tonsurans strains tested, Compound 8 clearly shows more potent antifungal activity as compared with fluconazole (Table 5a).

When tested in combination with fluconazole, Compound 8 showed synergism against 23% of the strains. No antagonism was demonstrated in the combination testing of these two drugs (Table 5b).

TABLE 4
MIC data (in μg/ml) of Compound 8, fluconazole, and terbinafine
Summary for T. rubrum strains

| | Fluconazole | Compound 8 | Terbinafine |
|---|---|---|---|
| Range (n = 20) | 0.25-4.0 | 0.5-4.0 | 0.008->8 |
| MIC₅₀ | 1 | 1 | 0.016 |
| MIC₉₀ | 1 | 4 | 8 |

Summary for T. mentagrophytes strains

| | Compound 8 | Fluconazole | Terbinafine |
|---|---|---|---|
| Range (n = 5) | 1.0 | 2.0-16.0 | 0.004-0.016 |
| MIC₅₀ | 1.0 | 16 | 0.008 |
| MIC₉₀ | 1.0 | 16 | 0.016 |

Summary for T. tonsurans strains

| | Compound 8 | Fluconazole | Terbinafine |
|---|---|---|---|
| Range (n = 5) | 0.06-0.12 | 1.0-4.0 | 0.001-0.016 |
| MIC₅₀ | 0.06 | 2.0 | 0.002 |
| M1C₉₀ | 0.06 | 4.0 | 0.002 |

TABLE 5-a
Raw Data
MIC Raw Data: Individual MIC Summary

| | | Fluconazole | Terbinafine | Compound |
|---|---|---|---|---|
| MRL # | Organism | (μg/ml) | (μg/ml) | 8 (μg/ml) |
| 475 | T. rubrum | 0.25 | 8 | 4 |
| 476 | T. rubrum | 0.25 | 8 | 0.5 |
| 477 | T. rubrum | 0.25 | 8 | 1 |
| 479 | T. rubrum | 0.5 | 8 | 4 |
| 670 | T. rubrum | 2 | 8 | 0.5 |
| 671 | T. rubrum | 4 | 4 | 2 |
| 1386 | T. rubrum | 0.25 | >8 | 2 |
| 9482 | T. rubrum | 0.5 | 4 | 4 |
| 12414 | T. rubrum | 1 | >8 | 4 |
| 17387 | T. rubrum | 0.5 | 0.016 | 1 |
| 17388 | T. rubrum | 1 | 0.016 | 1 |
| 17390 | T. rubrum | 1 | 0.016 | 1 |
| 17392 | T. rubrum | 1 | 0.016 | 1 |
| 17393 | T. rubrum | 1 | 0.016 | |
| 17395 | T. rubrum | 1 | 0.008 | 0.5 |
| 17396 | T. rubrum | 1 | 0.016 | 1 |
| 17434 | T. rubrum | 0.25 | 0.016 | 4 |
| 17435 | T. rubrum | 1 | 0.03 | 2 |
| 17436 | T. rubrum | 1 | 0.016 | 2 |
| 17437 | T. rubrum | 0.5 | 0.008 | 0.5 |
| 11256 | T. mentagrophytes | 16 | 0.016 | 1 |
| 12511 | T. mentagrophytes | 16 | 0.008 | 1 |
| 12679 | T. mentagrophytes | 8 | 0.008 | 1 |
| 12680 | T. mentagrophytes | 2 | 0.016 | 1 |
| 12692 | T. mentagrophytes | 16 | 0.004 | 1 |
| 10152 | T. tonsurans | 4 | 0.016 | 0.12 |
| 10245 | T. tonsurans | 1 | 0.002 | 0.06 |
| 10327 | T. tonsurans | 1 | 0.001 | 0.06 |
| 10326 | T. tonsurans | 4 | 0.002 | 0.06 |
| 10328 | T. tonsurans | 2 | 0.002 | 0.06 |

TABLE 5-b
Combination Raw Data

| | | Flucona- | Com- | | |
|---|---|---|---|---|---|
| | | zole | pound 8 | | |
| | | Combo | Combo | | |
| | | MIC | MIC | | Interpretation |
| MRL # | Organism | (μg/ml) | (μg/ml) | FIC | from ODDS |
| 475 | T. rubrum | 0.25 | 2 | 1.50 | No interaction |
| 476 | T. rubrum | 0.12 | 0.5 | 1.48 | No interaction |
| 477 | T. rubrum | 0.12 | 0.25 | 0.73 | Additive |
| 479 | T. rubrum | 0.5 | 0.06 | 1.02 | No interaction |
| 670 | T. rubrum | 0.12 | 0.06 | 0.18 | Synergistic |
| 671 | T. rubrum | 2 | 0.06 | 0.53 | Additive |
| 1386 | T. rubrum | 0.12 | 0.5 | 0.73 | Additive |
| 9482 | T. rubrum | 0.25 | 2 | 1.00 | Addivtive |
| 12414 | T. rubrum | 0.5 | 1 | 0.75 | Additive |
| 17387 | T. rubrum | 0.12 | 0.12 | 0.36 | Synergistic |
| 17388 | T. rubrum | 1 | 0.25 | 1.25 | No interaction |
| 17390 | T. rubrum | 0.25 | 0.12 | 0.37 | Synergistic |
| 17392 | T. rubrum | 0.5 | 0.25 | 0.75 | Additive |
| 17393 | T. rubrum | 0.25 | 0.25 | 0.50 | Additive |
| 17395 | T. rubrum | 0.25 | 0.06 | 0.37 | Synergistic |
| 17396 | T. rubrum | 0.25 | 0.25 | 0.50 | Additive |
| 17434 | T. rubrum | 0.06 | 1 | 0.49 | Synergistic |
| 17435 | T. rubrum | 0.25 | 0.5 | 0.50 | Additive |
| 17436 | T. rubrum | 0.25 | 0.5 | 0.50 | Additive |
| 17437 | T. rubrum | 0.25 | 0.06 | 0.62 | Additive |
| 11256 | T. mentagrophytes | 2 | 0.25 | 0.38 | Synergistic |
| 12511 | T. mentagrophytes | 4 | 0.5 | 0.75 | Additive |
| 12679 | T. mentagrophytes | 4 | 0.25 | 0.75 | Additive |
| 12680 | T. mentagrophytes | 0.015 | 1 | 1.01 | No interaction |
| 12692 | T. mentagrophytes | 8 | 0.06 | 0.56 | Additive |
| 10152 | T. tonsurans | 2 | 0.06 | 1.00 | Additive |
| 10245 | T. tonsurans | 0.25 | 0.03 | 0.75 | Additive |
| 10327 | T. tonsurans | 0.12 | 0.06 | 1.12 | No interaction |
| 10326 | T. tonsurans | 1 | 0.016 | 0.52 | Additive |
| 10328 | T. tonsurans | 0.25 | 0.016 | 0.39 | Synergistic |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for inhibiting the growth of a fungus or fungal unit thereof involved in infection and/or disease of keratinized tissue, comprising contacting the fungus or fungal unit thereof with a growth inhibiting effective amount of a compound of Formula (I) or Formula (II), wherein
the compounds of Formula (I) are represented by the formula:
Cy²-L²-Ar²—Y²—C(O)NH—Z (I)
and N-oxides, hydrates, solvates, tautomers, pharmaceutically acceptable salts, prodrugs and complexes thereof, and racemic and scalemic mixtures, diastereomers and enantiomers thereof, wherein
Cy² is H, cycloalkyl, aryl, heteroaryl, or heterocyclyl, any of which may be optionally substituted, provided that Cy² is not a (spirocycloalkyl)heterocyclyl;
L² is C₁-C₈ saturated alkylene or C₂-C₈ alkenylene, wherein the alkylene or alkenylene optionally may be substituted, and wherein one or two of the carbon atoms of the alkylene is optionally replaced by a heteroatomic moiety independently selected from the group consisting of O; NR′, R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted: and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z is selected from the group consisting of anilinyl, pyridyl, thiadiazolyl, each of which is optionally substituted, and —O-M, M being H or a pharmaceutically acceptable cation; and
the compounds of Formula (II) are represented by the formula: 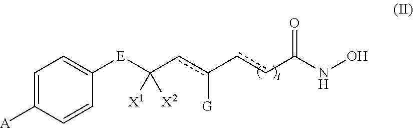
and N-oxides, hydrates, solvates, tautomers, pharmaceutically acceptable salts, prodrugs and complexes thereof, and racemic and scalemic mixtures, diastereomers and enantiomers thereof, wherein
A is selected from the group consisting of —O(CH₃), —NH₂ and aryl, wherein the aryl is optionally connected to the phenyl via a covalent bond or the aryl is fused to the phenyl;
E is selected from the group consisting of CH₂, CH(OCH₃), C═N(OH), C═CH₂ and O;
X¹ and X² are independently selected from the group consisting of H and CH₃;
G is selected from the group consisting of H and CH₃; [symbol]
is selected from the group consisting of a single bond and a double bond; and
t is an integer from 0 to 1,
with the proviso that the compound of formula (II) is not a compound selected from the group consisting of

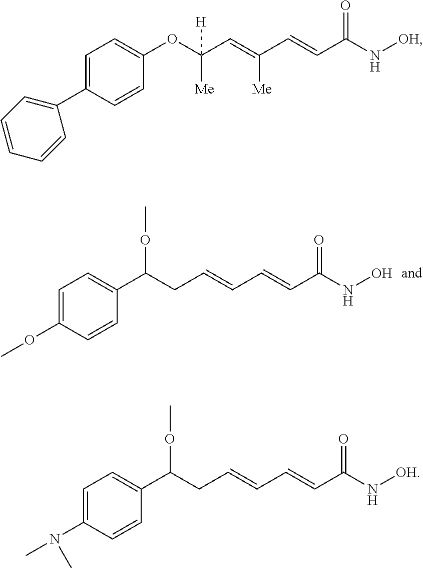

2. A method for inhibiting the growth of a fungus or fungal unit thereof involved in infection and/or disease of keratinized tissue, comprising inhibiting the activity of a histone deacetylase in the fungus or fungal unit thereof.

3. A method for treating and/or preventing a fungal infection and/or disease of keratinized tissue, in a subject comprising administering to the subject in need thereof a treatment or preventative effective amount of a compound of Formula (I) or Formula (II),
wherein
the compounds of Formula (I) are represented by the formula:
Cy²-L²-Ar²—Y²—C(O)NH—Z (I)
and N-oxides, hydrates, solvates, tautomers, pharmaceutically acceptable salts, prodrugs and complexes thereof, and racemic and scalemic mixtures, diastereomers and enantiomers thereof, wherein
Cy² is H, cycloalkyl, aryl, heteroaryl, or heterocyclyl, any of which may be optionally substituted, provided that Cy² is not a (spirocycloalkyl)heterocyclyl;
L² is C₁-C₈ saturated alkylene or C₂-C₈ alkenylene, wherein the alkylene or alkenylene optionally may be substituted, and wherein one or two of the carbon atoms of the alkylene is optionally replaced by a heteroatomic moiety independently selected from the group consisting of O; NR′, R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z is selected from the group consisting of anilinyl, pyridyl, thiadiazolyl, each of which is optionally substituted, and —O-M, M being H or a pharmaceutically acceptable cation; and
the compounds of Formula (II) are represented by the formula: 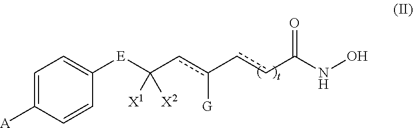
and N-oxides, hydrates, solvates, tautomers, pharmaceutically acceptable salts, prodrugs and complexes thereof, and racemic and scalemic mixtures, diastereomers and enantiomers thereof, wherein
A is selected from the group consisting of —O(CH₃), —NH₂ and aryl, wherein the aryl is optionally connected to the phenyl via a covalent bond or the aryl is fused to the phenyl;
E is selected from the group consisting of CH₂, CH(OCH₃), C═N(OH), C═CH₂ and O;
X¹ and X² are independently selected from the group consisting of H and CH₃;
G is selected from the group consisting of H and CH₃; [symbol]
is selected from the group consisting of a single bond and a double bond; and
t is an integer from 0 to 1,
with the proviso that the compound of formula (II) is not a compound selected from the group consisting of

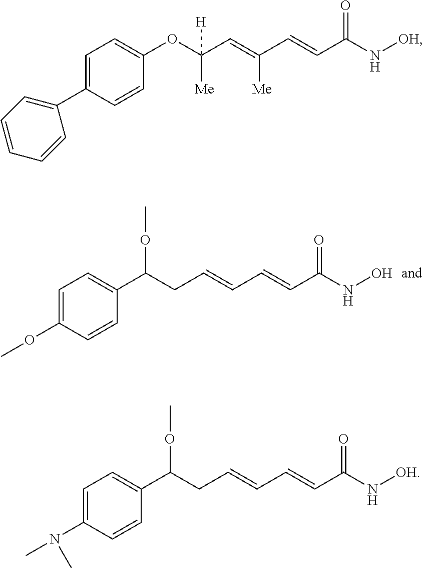

4. A method for treating and/or preventing a fungal infection and/or disease of keratinized tissue, in a subject, comprising administering to the subject in need thereof a treatment and/or preventative effective amount of an inhibitor of the activity of a histone deacetylase in the fungus or fungal unit thereof.

5. A method for sensitizing a fungus or fungal unit thereof involved in infection and/or disease of keratinized tissue, to an antifungal compound, comprising contacting the fungus or fungal unit thereof with a sensitizing effective amount of a compound of Formula (I) or Formula (II),
wherein
the compounds of Formula (I) are represented by the formula:
Cy²-L²-Ar²—Y²—C(O)NH—Z (I)
and N-oxides, hydrates, solvates, tautomers, pharmaceutically acceptable salts, prodrugs and complexes thereof, and racemic and scalemic mixtures, diastereomers and enantiomers thereof, wherein
Cy² is H, cycloalkyl, aryl, heteroaryl, or heterocyclyl, any of which may be optionally substituted, provided that Cy² is not a (spirocycloalkyl)heterocyclyl;
L² is C₁-C₈ saturated alkylene or C₂-C₈ alkenylene, wherein the alkylene or alkenylene optionally may be substituted, and wherein one or two of the carbon atoms of the alkylene is optionally replaced by a heteroatomic moiety independently selected from the group consisting of O; NR′, R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z is selected from the group consisting of anilinyl, pyridyl, thiadiazolyl, each of which is optionally substituted, and —O-M, M being H or a pharmaceutically acceptable cation; and
the compounds of Formula (II) are represented by the formula: 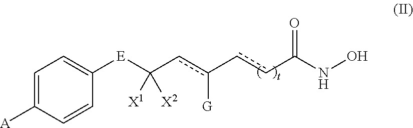
and N-oxides, hydrates, solvates, tautomers, pharmaceutically acceptable salts, prodrugs and complexes thereof, and racemic and scalemic mixtures, diastereomers and enantiomers thereof, wherein
A is selected from the group consisting of —O(CH₃), —NH₂ and aryl, wherein the aryl is optionally connected to the phenyl via a covalent bond or the aryl is fused to the phenyl;
E is selected from the group consisting of CH₂, CH(OCH₃), C═N(OH), C═CH₂ and O;
X¹ and X² are independently selected from the group consisting of H and CH₃;
G is selected from the group consisting of H and CH₃; [symbol]
is selected from the group consisting of a single bond and a double bond; and
t is an integer from 0 to 1,
with the proviso that the compound of formula (II) is not a compound selected from the group consisting of

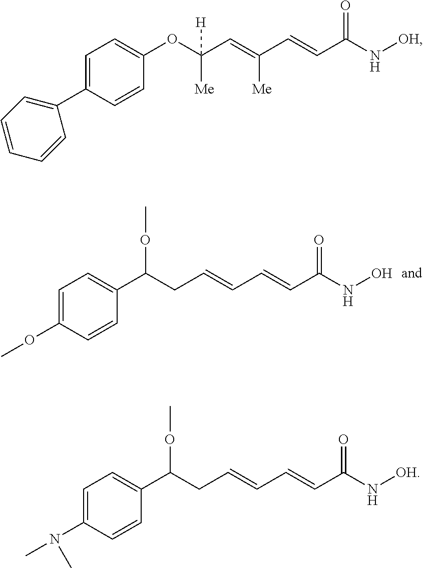

6. A method for sensitizing a fungus or fungal unit thereof involved in infection and/or disease of keratinized tissue, to an antifungal compound, comprising inhibiting the activity of a histone deacetylase in the fungus or fungal unit thereof.

7. A method for enhancing the activity of an antifungal agent against a fungus or fungal unit thereof involved in infection and/or disease of keratinized tissue, comprising contacting the fungus or fungal unit thereof with the antifungal agent in combination with an activity enhancing effective amount of a compound of Formula (I) or Formula (II),
wherein
the compounds of Formula (I) are represented by the formula:
Cy²-L²-Ar²—Y²—C(O)NH—Z (I)
and N-oxides, hydrates, solvates, tautomers, pharmaceutically acceptable salts, prodrugs and complexes thereof, and racemic and scalemic mixtures, diastereomers and enantiomers thereof, wherein
Cy² is H, cycloalkyl, aryl, heteroaryl, or heterocyclyl, any of which may be optionally substituted, provided that Cy² is not a (spirocycloalkyl)heterocyclyl;
L² is C₁-C₈ saturated alkylene or C₂-C₈ alkenylene, wherein the alkylene or alkenylene optionally may be substituted, and wherein one or two of the carbon atoms of the alkylene is optionally replaced by a heteroatomic moiety independently selected from the group consisting of O; NR′, R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z is selected from the group consisting of anilinyl, pyridyl, thiadiazolyl, each of which is optionally substituted, and —O-M, M being H or a pharmaceutically acceptable cation; and
the compounds of Formula (II) are represented by the formula: 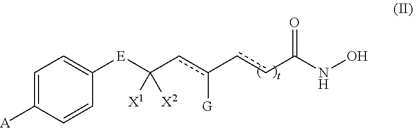
and N-oxides, hydrates, solvates, tautomers, pharmaceutically acceptable salts, prodrugs and complexes thereof, and racemic and scalemic mixtures, diastereomers and enantiomers thereof, wherein
A is selected from the group consisting of —O(CH₃), —NH₂ and aryl, wherein the aryl is optionally connected to the phenyl via a covalent bond or the aryl is fused to the phenyl;
E is selected from the group consisting of CH₂, CH(OCH₃), C═N(OH), C═CH₂ and O:
X¹ and X² are independently selected from the group consisting of H and CH₃;
G is selected from the group consisting of H and CH₃; [symbol]
is selected from the group consisting of a single bond and a double bond; and
t is an integer from 0 to 1,
with the proviso that the compound of formula (II) is not a compound selected from the group consisting of

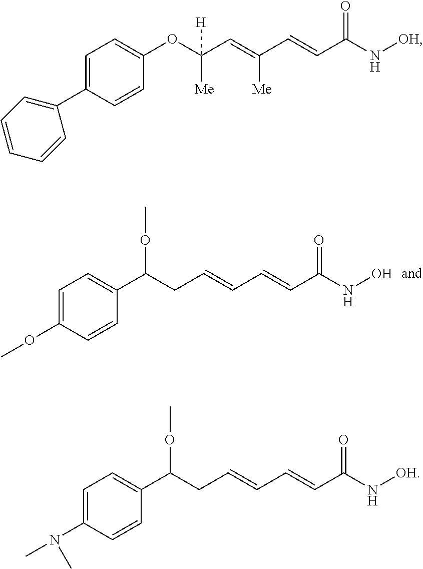

8. A method for enhancing the activity of an antifungal agent against a fungus or fungal unit thereof involved in infection and/or disease of keratinized tissue, comprising contacting the fungus or fungal unit thereof with the antifungal agent in combination with inhibiting the activity of a histone deacetylase in the fungus or fungal unit thereof.

9. The method according to claim 1 or claim 2, further comprising contacting the fungus or fungal unit thereof with another antifungal agent.

10. The method according to claim 3 or claim 4, further comprising administering to the subject another antifungal agent.

11. The method according to any of claims 1 to 10, wherein the compound is a compound of Formula (I).

12. The method according to any of claims 1 to 10, wherein the compound is a compound of Formula (II).

13. The method according to any of claims 1 to 10, wherein the compound is represented by the Formula (Ia): 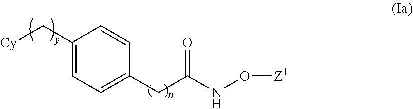
N-oxides, hydrates, solvates, tautomers, pharmaceutically acceptable salts, prodrugs and complexes thereof, and racemic and scalemic mixtures, diastereomers and enantiomers thereof, wherein
Cy is alkyl, cycloalkyl, aryl, heteroaryl or heterocyclyl, any of which maybe optionally substituted;
x is an integer from 0 to 5, wherein the chain of length x is optionally substituted and wherein one or two carbon atoms of the chain of length x is optionally replaced with a heteroatom;
n is an integer from 0 to 2; and
Z¹ is selected from the group consisting of H and a heterocyclic group;
with the provisos that when x is 4, n is not 2, and when x is 3, n is not 3.

14. The method of claim 13, wherein the compound is selected from the group consisting of 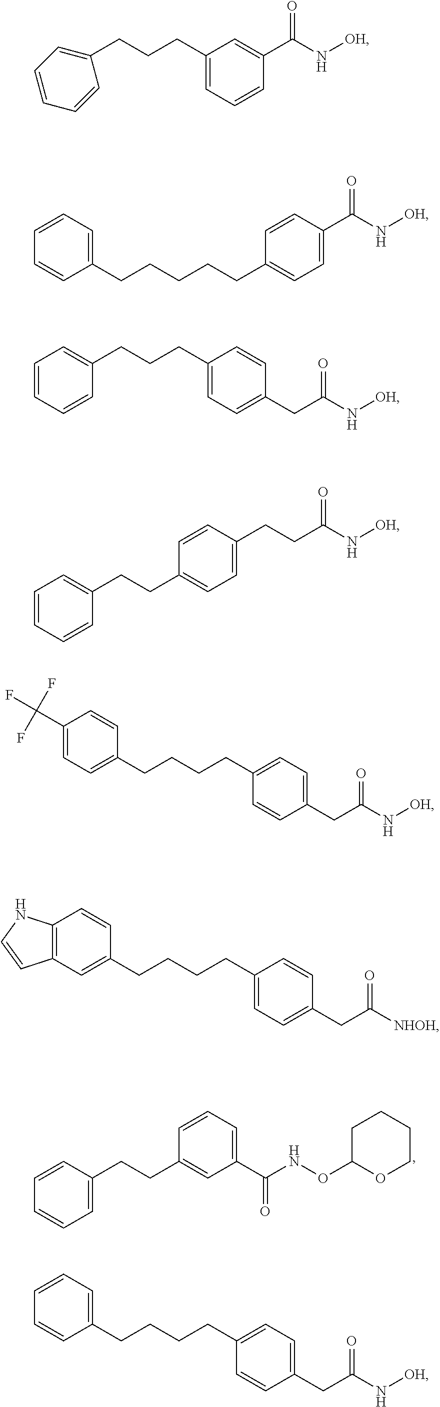 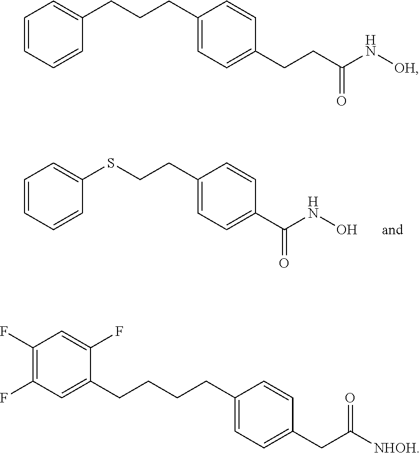
and N-oxides, hydrates, solvates, tautomers, pharmaceutically acceptable salts, prodrugs and complexes thereof, and racemic and scalemic mixtures, diastereomers and enantiomers thereof.

15. The method of claim 13, wherein the compound is selected from the group consisting of 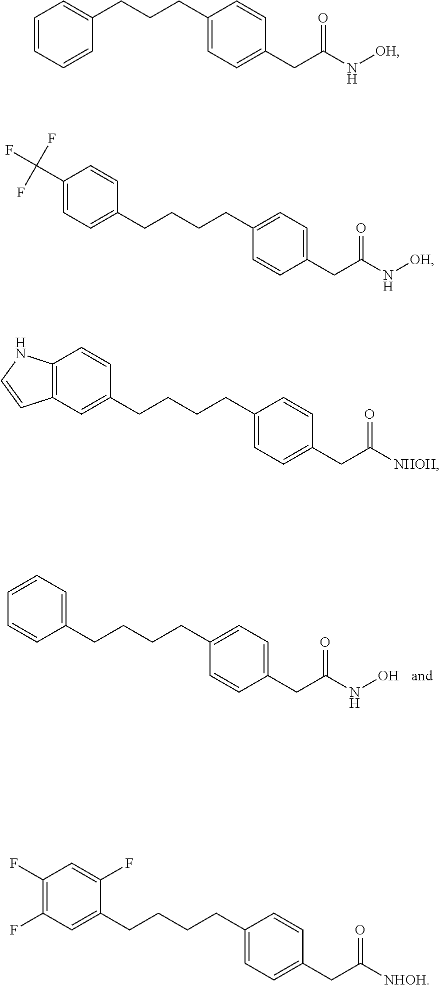
and N-oxides, hydrates, solvates, tautomers, pharmaceutically acceptable salts, prodrugs and complexes thereof, and racemic and scalemic mixtures, diastereomers and enantiomers thereof.

16. The method of claim 13, wherein, the compound is 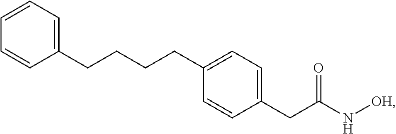
or a hydrate, solvate, tautomer, pharmaceutically acceptable salt, prodrug or complex thereof, and racemic and scalemic mixtures, diastereomers and enantiomers thereof.

17. The method according to any of claims 1 to 10, wherein the compound is selected from the group consisting of 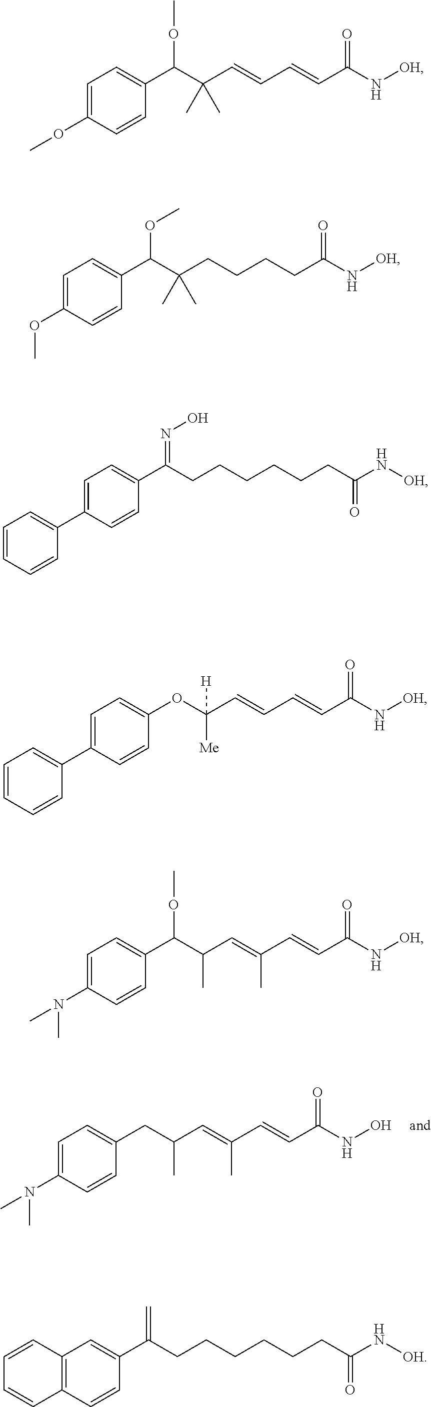
and N-oxides, hydrates, solvates, tautomers, pharmaceutically acceptable salts, prodrugs and complexes thereof, and racemic and scalemic mixtures, diastereomers and enantiomers thereof.

18. The method according to any of claims 1 to 10, wherein the compound is 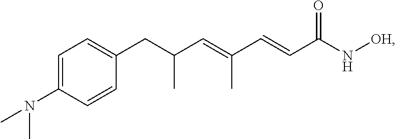
or an N-oxide, hydrate, solvate, tautomer, pharmaceutically acceptable salt, prodrug or complex thereof, and racemic and scalemic mixtures, diastereomers and enantiomers thereof.

19. A kit, comprising a compound of Formula (I) or Formula (II), and optionally instructions for using the kit in a method according to any of claim 1, 3, 5 or 7
wherein
the compounds of Formula (I) are represented by the formula:
Cy²-L²-Ar²—Y²—C(O)NH—Z (I)
and N-oxides, hydrates, solvates, tautomers, pharmaceutically acceptable salts, prodrugs and complexes thereof, and racemic and scalemic mixtures, diastereomers and enantiomers thereof, wherein
Cy² is H, cycloalkyl, aryl, heteroaryl, or heterocyclyl, any of which may be optionally substituted, provided that Cy² is not a (spirocycloalkyl)heterocyclyl;
L² is C₁-C₈ saturated alkylene or C₂-C₈ alkenylene, wherein the alkylene or alkenylene optionally may be substituted, and wherein one or two of the carbon atoms of the alkylene is optionally replaced by a heteroatomic moiety independently selected from the group consisting of O; NR′, R′ being alkyl, acyl, or hydrogen; S; S(O); or S(O)₂;
Ar² is arylene, wherein said arylene optionally may be additionally substituted and optionally may be fused to an aryl or heteroaryl ring, or to a saturated or partially unsaturated cycloalkyl or heterocyclic ring, any of which may be optionally substituted; and
Y² is a chemical bond or a straight- or branched-chain saturated alkylene, which may be optionally substituted, provided that the alkylene is not substituted with a substituent of the formula —C(O)R wherein R comprises an α-amino acyl moiety; and
Z is selected from the group consisting of anilinyl, pyridyl, thiadiazolyl, each of which is optionally substituted, and —O-M, M being H or a pharmaceutically acceptable cation; and
the compounds of Formula (II) are represented by the formula: 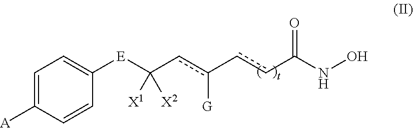
and N-oxides, hydrates, solvates, tautomers, pharmaceutically acceptable salts, prodrugs and complexes thereof, and racemic and scalemic mixtures, diastereomers and enantiomers thereof, wherein
A is selected from the group consisting of —O(CH₃), —NH₂ and aryl, wherein the aryl is optionally connected to the phenyl via a covalent bond or the aryl is fused to the phenyl;
E is selected from the group consisting of CH₂, CH(OCH₃), C═N(OH), C═CH₂ and O;
X¹ and X² are independently selected from the group consisting of H and CH₃;
G is selected from the group consisting of H and CH₃; [symbol]
is selected from the group consisting of a single bond and a double bond; and
t is an integer from 0 to 1,
with the proviso that the compound of formula (II) is not a compound selected from the group consisting of

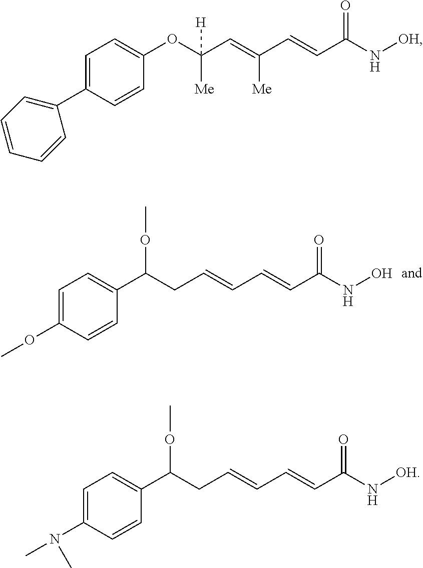

20. A kit, comprising an inhibitor of fungal histone deacetylase, and optionally instructions for using the kit in a method according to any of claim 2, 4, 6 or 8.

21. A composition comprising a combination of a compound of Formula (I) or Formula (II) or an N-oxide, hydrate, solvate, tautomer, pharmaceutically acceptable salt, prodrug or complex thereof, and racemic and scalemic mixtures, diastereomers and enantiomers thereof, as a component (I) together with an antifungal agent, as a component (II); whereby component (I) and component (II) are in respective proportions to provide a synergistic effect against a fungus or fungal unit thereof involved in infection and/or disease of keratinized tissue.
